(19) United States
(12) Patent Application Publication
(10) Pub. No.: US 2016/0120514 A1
(43) Pub. Date: May 5, 2016

(54) ULTRASONIC MEASUREMENT DEVICE, ULTRASONIC IMAGING DEVICE, AND ULTRASONIC MEASUREMENT METHOD

(71) Applicant: SEIKO EPSON CORPORATION, Tokyo (JP)

(72) Inventor: Takumi ARAGAKI, Matsumoto (JP)

(21) Appl. No.: 14/920,342

(22) Filed: Oct. 22, 2015

(30) Foreign Application Priority Data
Oct. 31, 2014 (JP) 2014-222251

(51) Int. Cl. A61B 8/08; A61B 8/00; A61B 8/14

(52) U.S. Cl. CPC A61B 8/5215; A61B 8/5207; A61B 8/14; A61B 8/461

(57) ABSTRACT

An ultrasonic measurement device 100 includes a transmission processing unit 110 that performs processing for transmitting an ultrasonic wave to a subject, a reception processing unit 120 that performs processing for receiving an ultrasonic echo reflected by the transmitted ultrasonic wave, and a processing unit 130 that performs processing on a received signal from the reception processing unit 120. The processing unit 130 performs, on the received signal, processing for specifying the reflection intensities from point scatterers in the subject based on M basis waves (where M is an integer of 2 or more) at least two of which have phases shifted from each other with a phase difference smaller than a phase difference that corresponds to the pulse width of a transmission pulse signal transmitted from the transmission processing unit 110 or to the pulse width of a received signal in response to the transmission pulse signal.

### BACKGROUND

1. Technical Field

The present invention relates to an ultrasonic measurement device, an ultrasonic imaging device, an ultrasonic measurement method, and the like.

2. Related Art

As a device for use in examining the inside of a human body that serves as a test subject, an ultrasonic measurement device that emits an ultrasonic wave toward the subject and receives reflective waves from interfaces of objects having different acoustic impedances inside the subject draws an attention. The ultrasonic measurement device is also applied to diagnostic imaging that is performed on a superficial layer of the test subject to measure the visceral fat, the blood flow rate, and the like.

When performing diagnostic imaging using such an ultrasonic measurement device, high resolution image processing of an ultrasonic echo needs to be performed and thus, for example, harmonic imaging (a harmonic imaging method) and the like are used.

Here, in the harmonic imaging, it is needed to extract a harmonic component of the ultrasonic echo, and corresponding methods for extracting the harmonic component include a filter method and a phase inversion method disclosed in JP-A-2002-360569 and the like. JP-A-2002-360569 discloses an ultrasonic imaging device that performs a phase inversion method using a high-order harmonic, namely, the third harmonic or higher harmonic.

In B-mode image generation processing in the related art, the distance resolution and the azimuth resolution are substantially the same, and thus an improvement in the distance resolution is not addressed as a problem. However, when harmonic imaging or adaptive beam forming is applied, the azimuth resolution is higher than the distance resolution, and a generated image newly has an anisotropy in the resolution. For example, also in the above-described JP-A-2002-360569, the distance resolution is relatively reduced as compared with the azimuth resolution. Therefore, an improvement in the distance resolution is needed.

### SUMMARY

According to some aspects of the invention, it is possible to provide an ultrasonic measurement device, an ultrasonic imaging device, an ultrasonic measurement method, and the like that can improve not only the azimuth resolution but also the distance resolution of a subject measurement result using an ultrasonic wave.

According to an aspect of the invention, an ultrasonic measurement device includes: a transmission processing unit that performs processing for transmitting an ultrasonic wave to a subject; a reception processing unit that performs processing for receiving an ultrasonic echo of the transmitted ultrasonic wave; and a processing unit that performs processing on a received signal from the reception processing unit, wherein the processing unit performs, on the received signal, processing for specifying reflection intensities from point scatterers in the subject based on M basis waves (where M is an integer of 2 or more) at least two of which have phases shifted from each other with a phase difference smaller than a phase difference that corresponds to a pulse width of a transmission pulse signal transmitted by the transmission processing unit or a pulse width of a received signal in response to the transmission pulse signal.

According to the aspect of the invention, the received signal is subjected to processing for specifying reflection intensities from point scatterers in the subject based on M basis waves (where M is an integer of 2 or more) that have phases shifted from each other with a phase difference smaller than a phase difference that corresponds to the pulse width of the transmission pulse signal or the pulse width of the received signal. Accordingly, it is possible to improve not only the azimuth resolution but also the distance resolution of a subject measurement result using the ultrasonic wave.

Furthermore, according to the aspect of the invention, it is preferable that a storage unit in which respective pieces of basis wave data on the M basis waves are stored in association with M measurement points that are set in a depth direction of the subject be provided.

Accordingly, it is possible, for example, to read respective pieces of basis wave data on the M basis waves from the storage unit (memory), and to decompose the received wave into M basis wave components.

Furthermore, according to the aspect of the invention, it is preferable that a phase difference between an i-th basis wave (where i is an integer of 1≦i≦M) and an (i+1)-th basis wave of the M basis waves be smaller than a phase distance that corresponds to the pulse width of the transmission pulse signal or the pulse width of the received signal.

Accordingly, the received wave with which backscattered waves from a plurality of scatterers interfered is decomposed into basis waves before the interference that have changed phases, and thus it is possible, for example, to provide a high quality image having a distance resolution higher than the distance resolution determined depending on the pulse width of the ultrasonic wave.

Furthermore, according to the aspect of the invention, it is preferable that the i-th basis wave (where i is an integer of 1≦i≦M) of the M basis waves be a waveform that corresponds to a received signal of the ultrasonic wave from an i-th point scatterer arranged at an i-th measurement point, and the (i+1)-th basis wave of the M basis waves be a waveform that corresponds to a received signal of the ultrasonic wave from an (i+1)-th point scatterer arranged at an (i+1)-th measurement point, which is farther away from a transmission point of the ultrasonic wave than the i-th measurement point.

Accordingly, it is possible, for example, to extract basis wave components from the received wave with a distance resolution that corresponds to a set distance between the measurement points.

Furthermore, according to the aspect of the invention, it is preferable that the processing unit obtain the reflection intensities by performing deconvolution processing of the M basis waves on the received signal.

Accordingly, it is possible, for example, to specify the position of each scatterer in the subject based on the reflection intensities.

Furthermore, according to the aspect of the invention, it is preferable that a set distance between measurement points in a first depth range of the subject be smaller than a set distance between measurement points in a second depth range of the subject.

Accordingly, it is possible, for example, to reduce the number of the basis waves while improving the distance resolution in the first depth range of the subject.

Furthermore, according to the aspect of the invention, it is preferable that the processing unit perform the processing for specifying reflection intensities in the first depth range of the subject, and do not perform the processing for specifying reflection intensities in the second depth range of the subject.

Accordingly, it is possible, for example, to generate a B-mode image in which only the depth range of the subject that is needed to be measured is imaged, and to reduce the processing load of the ultrasonic measurement device.

Furthermore, according to the aspect of the invention, it is preferable that the processing unit perform processing for generating a B-mode image based on the reflection intensities specified by the specifying processing.

Accordingly, it is possible, for example, to display on the display unit an image that makes a user easily understandable the state of the inside of the subject.

Furthermore, according to the aspect of the invention, it is preferable that each of the M basis waves be a waveform obtained by shifting a phase of the pulse wave of the transmission pulse signal.

Accordingly, it is possible, for example, to generate a basis wave without performing ultrasonic wave transmission/reception processing.

Furthermore, according to another aspect of the invention, an ultrasonic imaging device includes the ultrasonic measurement device, and a display unit that displays image data for display that is created based on the reflection intensities.

Moreover, according to yet another aspect of the invention, an ultrasonic measurement method includes: performing processing for transmitting an ultrasonic wave to a subject; performing processing for receiving an ultrasonic echo reflected by the transmitted ultrasonic wave so as to obtain a received signal; and performing, on the received signal, processing for specifying reflection intensities from point scatterers in the subject based on M basis waves (where M is an integer of 2 or more) that have phases shifted from each other with a phase difference smaller than a phase difference that corresponds to a pulse width of a transmission pulse signal transmitted by the transmission processing unit or a pulse width of a received signal in response to the transmission pulse signal.

### BRIEF DESCRIPTION OF THE DRAWINGS

The invention will be described with reference to the accompanying drawings, wherein like numbers reference like elements.

FIG. 1 illustrates a filter method.

FIGS. 2A to 2C illustrate a phase inversion method.

FIGS. 3A and 3B illustrate processing in which the phase inversion method and the filter processing are used together.

FIGS. 4A and 4B illustrate the relationship between the distance resolution and the pulse width.

FIG. 5 illustrates an example of a configuration of a system according to a first embodiment.

FIG. 6 illustrates an example of a detailed system configuration of an ultrasonic imaging device according to the first embodiment.

FIGS. 7A to 7C illustrate specific examples of device configurations of the ultrasonic measurement device.

FIGS. 8A to 8C are diagrams illustrating an overview of processing of the first embodiment.

FIG. 9 is a flowchart illustrating the flow of overall processing of the first embodiment.

FIG. 10 is a flowchart illustrating the flow of processing for specifying the reflection intensity.

FIG. 11 is a flowchart illustrating the flow of processing for generating a basis wave.

FIGS. 12A to 12C illustrate the correspondence relationship between point scatterers and basis waves.

FIG. 13 illustrates a measurement result.

FIG. 14 illustrates an example of a system configuration of an ultrasonic imaging device according to a second embodiment.

FIGS. 15A to 15D illustrate processing for generating a reconstructed wave.

FIG. 16 is a flowchart illustrating the flow of the overall processing of the second embodiment.

FIG. 17 is a flowchart illustrating the flow of the processing for generating a reconstructed wave.

FIG. 18 is a flowchart illustrating the flow of processing for generating a first basis wave and a second basis wave.

FIGS. 19A to 19C illustrate in detail the processing for generating a second basis wave.

FIGS. 20A to 20C illustrate the correspondence relationship between point scatterers and the first and second basis waves.

FIGS. 21A and 21B illustrate a measurement result.

FIGS. 22A to 22C illustrate examples of a configuration of an ultrasonic transducer element.

FIG. 23 illustrates an example of a configuration of an ultrasonic transducer device.

FIGS. 24A and 24B illustrate examples of a configuration of an ultrasonic transducer element group that is provided for each channel.

### DESCRIPTION OF EXEMPLARY EMBODIMENTS

Hereinafter, embodiments of the invention will be described. Note that the embodiments that will be described below do not unduly limit the content of the invention recited in Claims. Furthermore, all configurations that will be described in the embodiments are not necessarily essential components of the invention.

### 1. Overview

As a device for use in examining the inside of a human body that serves as a test subject, an ultrasonic measurement device that emits an ultrasonic wave toward the subject and receives reflective waves from interfaces of objects having different acoustic impedances inside the subject is known. Furthermore, application examples of the ultrasonic measurement device include a pocket-type ultrasonic viewer and the like that perform diagnostic imaging on a superficial layer of the test subject to measure the visceral fat, the blood flow rate, and the like, and development of the ultrasonic measurement device in the health-care field is expected.

As described above, when performing diagnostic imaging using the ultrasonic measurement device, high resolution image processing on an ultrasonic echo is needed. A technique for realizing the high resolution image processing includes harmonic imaging (a harmonic imaging method).

The harmonic imaging refers to a method for imaging a harmonic component, which will be described later. Here, the speed of an ultrasonic wave (compressional wave) that is propagated in a medium is likely to be high in its portion in which a sound pressure is high, and to be low in its portion in which a sound pressure is low. Accordingly, even in a case of a simple sine wave, its waveform gradually deforms and changes in the propagation process, and includes a harmonic component (also referred to as a nonlinear component) having a frequency of an integral multiple of a basic frequency, the harmonic component being not included in the fundamental. Such a nonlinear effect is significant in proportion to the square of a sound pressure of an ultrasonic wave, and is accumulated in proportion to the propagation distance.

Also, harmonic imaging is broadly classified into two categories, namely, tissue harmonic imaging for imaging a harmonic component that occurs from tissue itself when an ultrasonic wave propagates in the tissue, and contrast harmonic imaging for imaging a harmonic component that occurs when microbubbles of an ultrasonic contrast agent resonate and collapse. In the embodiments, the tissue harmonic imaging is used.

Furthermore, the harmonic imaging has two advantages. First, the amplitude of a harmonic component has characteristics of being proportional to the square of the amplitude of a transmitted ultrasonic wave, and thus the amplitude of the harmonic component is high in the center of a transmitted beam in which a sound pressure is high, but becomes drastically lower toward the end from the beam center. Accordingly, in the harmonic imaging, the range in which a nonlinear effect occurs is restricted to the beam center, leading to an improvement in the azimuth resolution as compared with the other methods. This is the first advantage.

Furthermore, main noise that occurs in an ultrasonic image includes noise due to multiple reflection and noise due to a side lobe. Here, a reflected ultrasonic echo has a low sound pressure, and thus a harmonic component itself does not occur. Therefore, the noise due to multiple reflection is reduced. Furthermore, a side lobe has a low sound pressure, and thus a harmonic component itself does not occur also in the side lobe. Therefore, noise due to a side lobe is also reduced. In the harmonic imaging, it is thus possible to reduce both the noise due to multiple reflection and the noise due to a side lobe. This is the second advantage.

In the embodiments, among the harmonic imaging, third harmonic imaging for imaging a third harmonic component is performed. In the third harmonic imaging, the azimuth resolution can further be improved because the beam width is smaller than that in a method for imaging a second harmonic component.

Here, in the third harmonic imaging, it is needed to extract a third harmonic component of an ultrasonic echo, and corresponding extracting methods include a filter method and a phase inversion method.

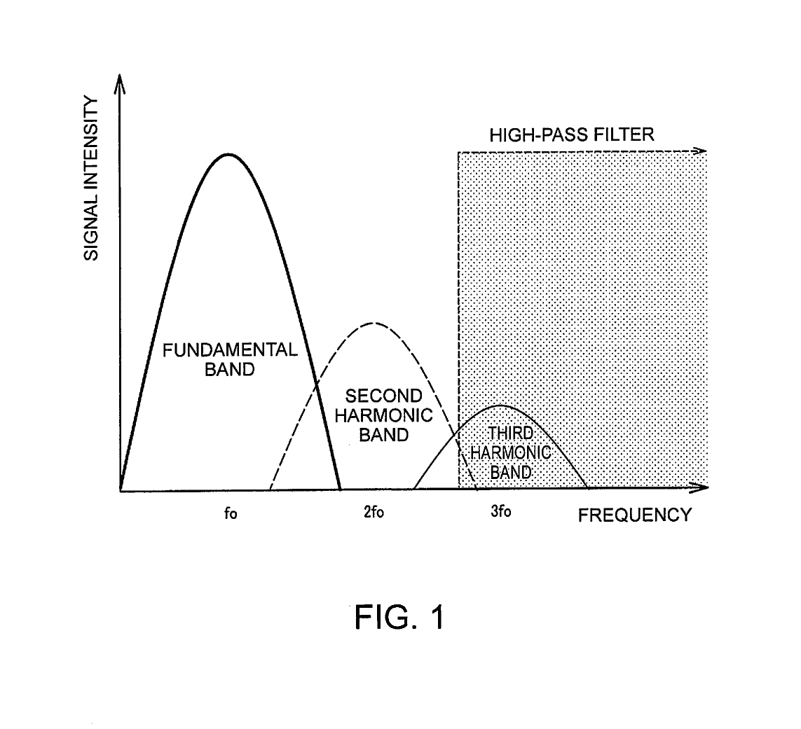

First, the filter method refers to a method in which a fundamental component and a second harmonic component are separated from a third harmonic component using a frequency filter (high-pass filter), and only the third harmonic component is extracted and imaged. For example, as a diagram for illustrating the filter method, FIG. 1 shows a received signal that has the fundamental band central frequency of f₀, the second harmonic band central frequency of 2f₀, and the third harmonic band central frequency of 3f₀ in a graph in which the vertical axis indicates the signal intensity and the horizontal axis indicates the frequency. Actually, as shown in FIG. 1, because the received fundamental component, second harmonic component, and third harmonic component each have a bandwidth, the second harmonic component and the third harmonic component overlap each other and cannot be separated from each other, resulting in image deterioration. In order to reduce this overlap, it is needed to elongate the pulse width. However, the long pulse width reduces the distance resolution.

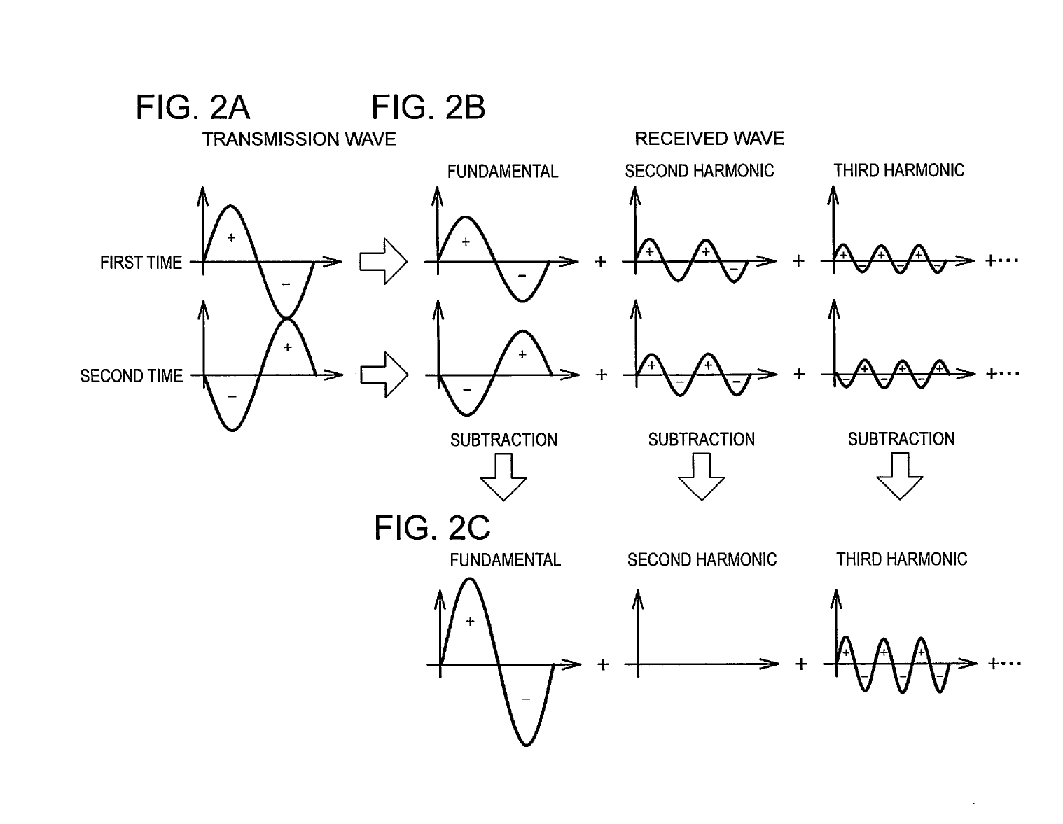

On the other hand, the phase inversion method is a method that is developed in order to improve defects of the filter method. In the phase inversion method, ultrasonic waves are transmitted twice one after another in the same direction. As shown in FIG. 2A, the second transmission wave has a phase different from that of the first transmission wave by 180 degrees.

Also, a received wave that was reflected on and returned from the biological body or a contrast agent includes a harmonic component due to its nonlinear propagation characteristics and thus has a deformed waveform. FIG. 2B shows received waves in response to the first and second transmission waves that is decomposed into a fundamental, a second harmonic, and a third harmonic. As shown in FIG. 2B, the two received waves have the relationship that the fundamental component and an odd harmonic component (third harmonic component) are inverted since the first and the second transmission waves are inverted, but an even harmonic component (second harmonic component) is not inverted. That is, with respect to the two received waves in response to the two transmission waves, phases of the fundamental component and an odd harmonic component are inverted, but the phase of an even harmonic component is the same.

Therefore, as shown in FIG. 2C, by performing subtraction of the two received waves, the second harmonic component is removed, and the fundamental component and the third harmonic component remain with their amplitude twofold. Accordingly, it is possible to extract the fundamental component and the third harmonic component.

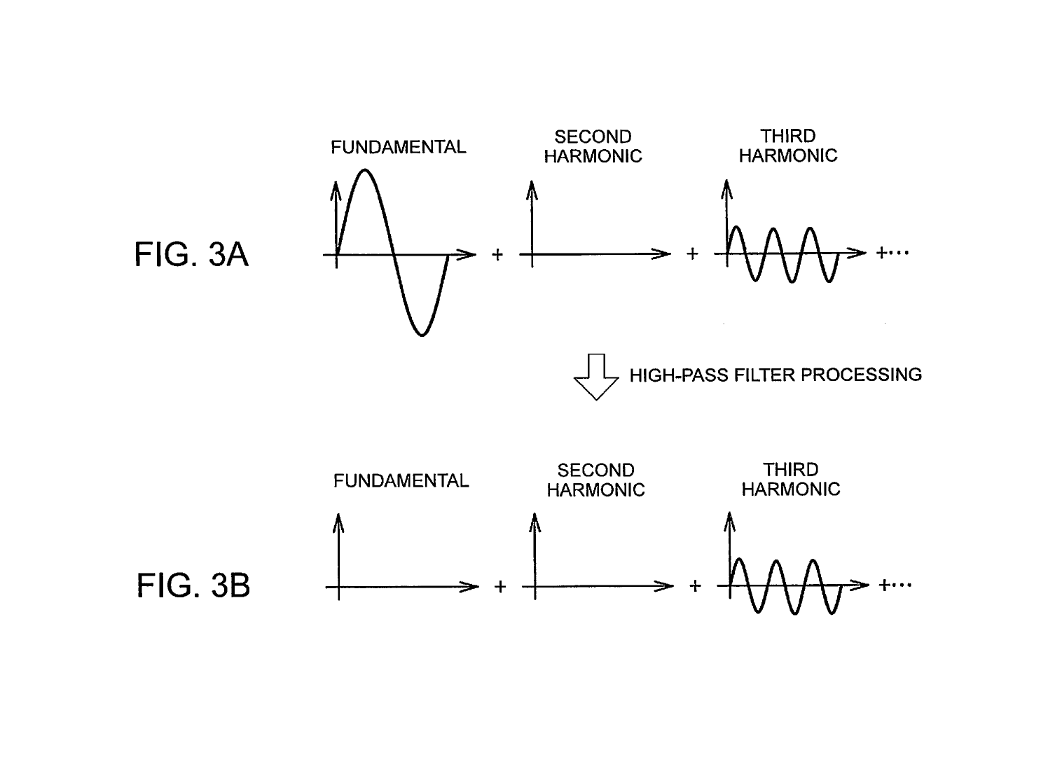

Furthermore, when only the target N-th harmonic component (where N is an integer of 2 or more) is extracted from the fundamental component, the odd harmonic component, or the even harmonic component that is extracted using the phase inversion method, it is necessary to perform the phase inversion method in combination with the above-described filter method. In the embodiments, the fundamental component and the third harmonic component that are shown in FIG. 3A and extracted using the phase inversion method are separated from each other using a frequency filter (high-pass filter or a band-pass filter) and only the third harmonic component is extracted and imaged as shown in FIG. 3B.

Accordingly, using the phase inversion method and the filter method in combination with each other, it is possible to generate a high quality B-mode image that has reduced artifacts due to a side lobe or multiple reflection and the improved azimuth resolution, as compared with the conventional method for generating a B-mode image only from a fundamental component.

However, even when a B-mode image is generated using the above-described method, the distance resolution cannot be improved. The distance resolution Δx is defined depending on the pulse width, and is obtained by the below formula (1). Note that in the below formula (1), n is a wavenumber(number of cycles) and λ is a wavelength.

$$\begin{matrix}Formula ( 1 ) & \\ Δ x = \frac{n λ}{2} & ( 1 )\end{matrix}$$

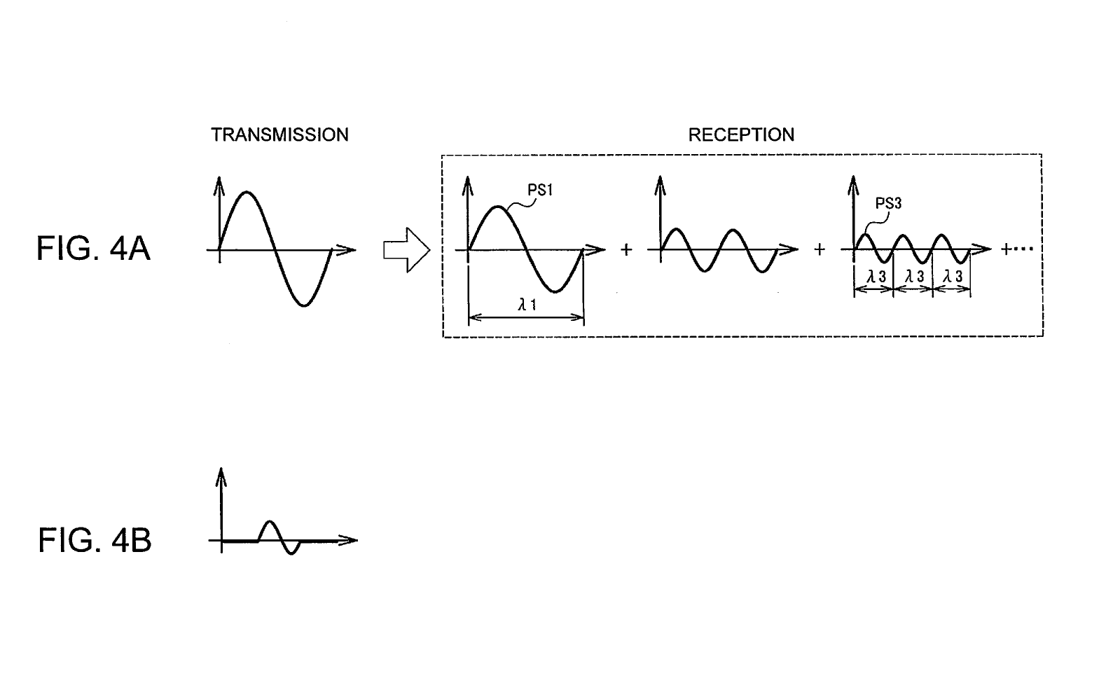

For example, in the case of the received wave in response to a transmission wave shown in FIG. 4A, a wavelength λ3 of a third harmonic component PS3 is one third of a wavelength λ1 of a fundamental component PS1, but a wavenumber n3 of the third harmonic component PS3 is threefold as large as a wavenumber nl of the fundamental component PS1. Accordingly, even by generating an image using the third harmonic component, the distance resolution Δx is the same as that in the case where the fundamental component is used. As shown in FIG. 4B, if a wave whose wavelength and wavenumber are smaller than those of the fundamental component can be used, it is possible to improve the distance resolution.

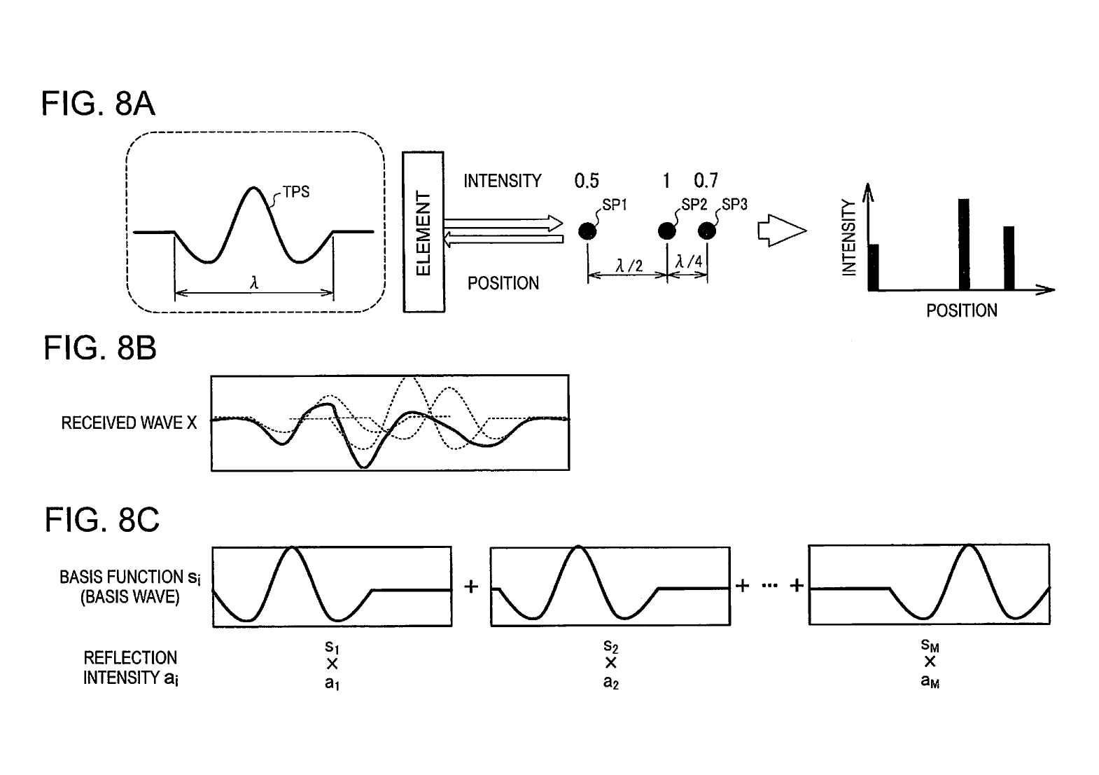

Accordingly, in a first embodiment, which will be described below, a received signal is subjected to processing for specifying the reflection intensities from point scatterers in the subject based on M basis waves (where M is an integer of 2 or more) at least two of which have phases shifted from each other with a phase difference smaller than a phase difference corresponding to the pulse width of a transmission pulse signal or the pulse width of the received signal. That is, in the first embodiment, a received wave X as shown in FIG. 8B, which will be described later, is decomposed into basis waves (basis functions) as shown in FIG. 8C, and a scatterer density distribution in the subject is specified. Accordingly, since the received wave with which backscattered waves from a plurality of scatterers interfered is decomposed into fundamentals before the interference that have changed phases, it is possible to provide a high quality image that has a distance resolution higher than that determined depending on the ultrasonic pulse width.

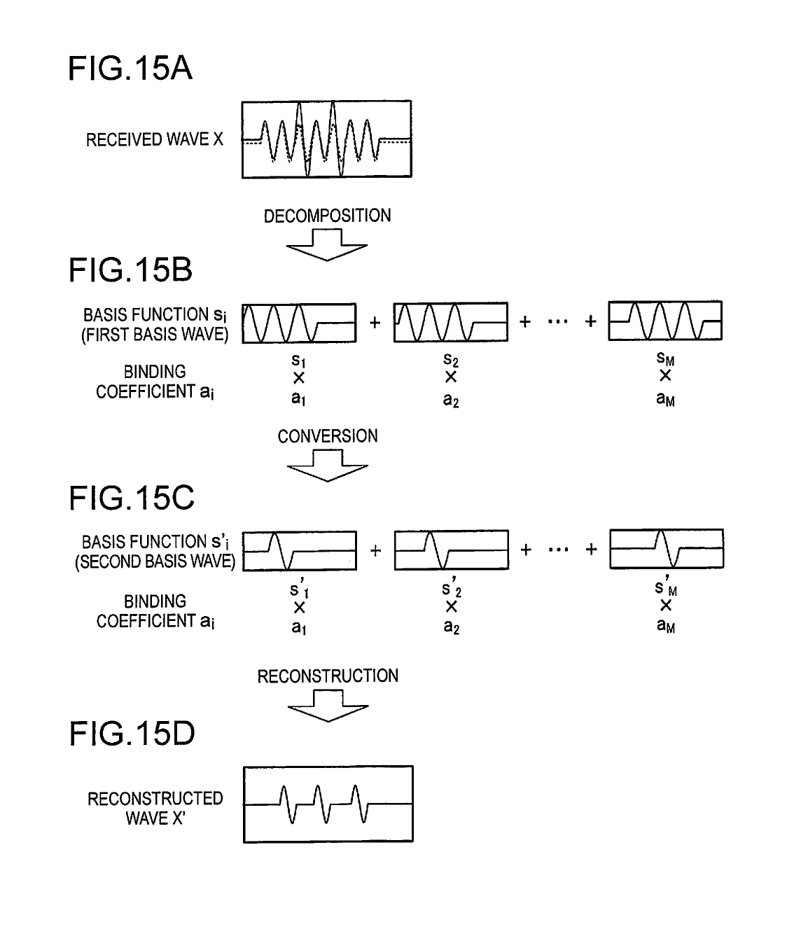

Furthermore, in a second embodiment, a received signal of an ultrasonic wave is subjected to processing for specifying binding coefficients of a plurality of first basis waves constituting a received signal, and then to processing for converting the received signal into a reconstructed signal based on the plurality of specified binding coefficients and the second basis wave whose wavenumber is smaller than that of the first basis wave. That is, in the second embodiment, the received wave X as shown in FIG. 15A, which will be described later, is reconstructed, and a reconstructed wave X′ shown in FIG. 15D is generated. This reconstructed wave X′ is constituted by second basis waves shown in FIG. 15C. The second basis waves have the same wavelength as and a smaller wavenumber than the first basis waves of FIG. 15B that constitutes the original received wave X. Accordingly, it is possible to improve the distance resolution.

### 2. First Embodiment

2.1. System Configuration Example

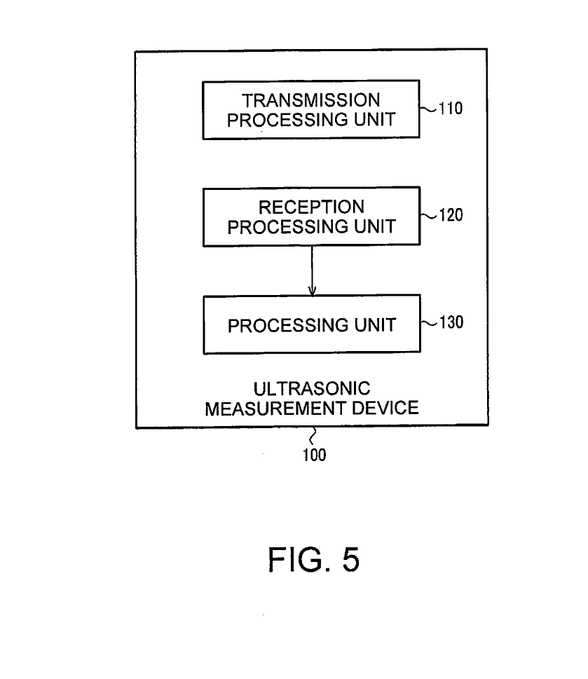

Hereinafter, an example of a configuration of an ultrasonic measurement device according to the present embodiment is shown in FIG. 5. An ultrasonic measurement device 100 includes a transmission processing unit 110, a reception processing unit 120, and a processing unit 130.

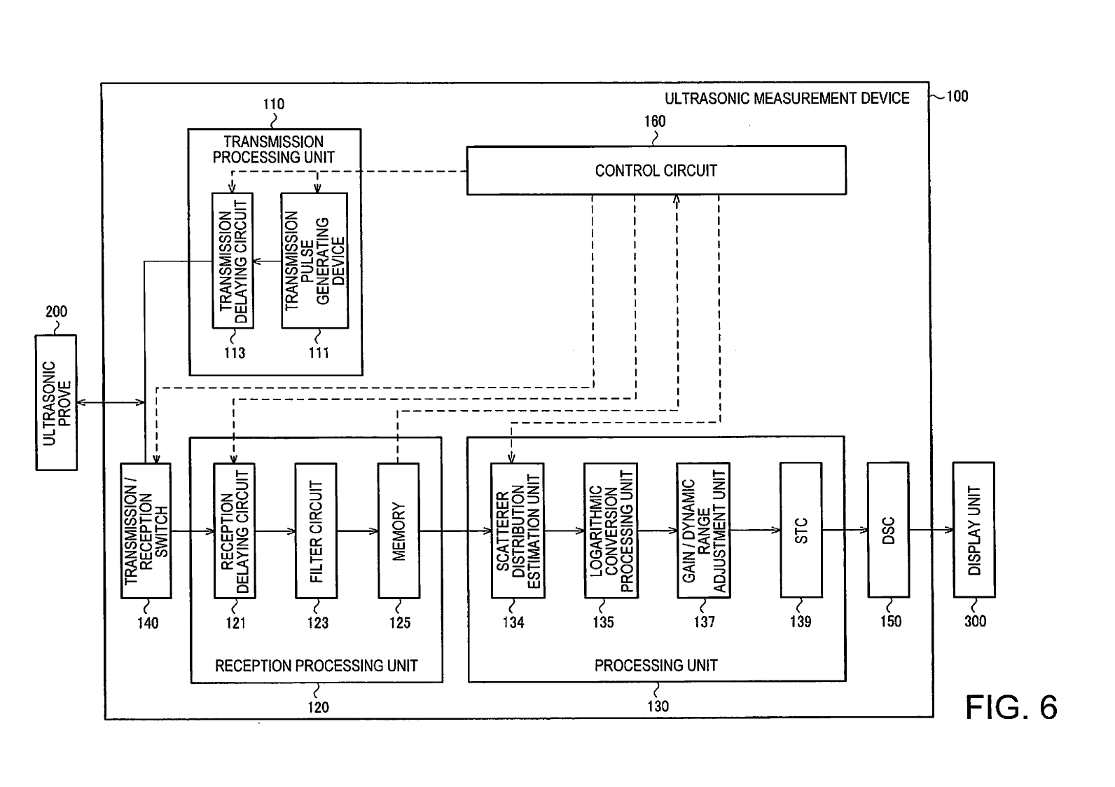

Furthermore, a specific example of a configuration of an ultrasonic imaging device according to the present embodiment is shown in FIG. 6. The ultrasonic imaging device includes the ultrasonic measurement device 100, an ultrasonic prove 200, and a display unit 300. Furthermore, the ultrasonic measurement device 100 shown in FIG. 6 includes the transmission processing unit 110, the reception processing unit 120, the processing unit 130, a transmission/reception switch 140, a digital scan convertor (DSC) 150, and a control circuit 160.

Note that the ultrasonic measurement device 100 and the ultrasonic imaging device including this are not limited to the configuration of FIGS. 5 and 6, and various modifications can be executed, such as one in which some of these constituent components are omitted, or one in which another constituent component is added. Furthermore, some or all of the functions of the ultrasonic measurement device 100 and the ultrasonic imaging device including this according to the present embodiment may be realized by a server connected thereto via communication.

Hereinafter, processing that is performed in the constituent components will be described.

The ultrasonic prove 200 includes an ultrasonic transducer device.

Also, the ultrasonic transducer device transmits an ultrasonic beam to a subject while the subject is scanned along the scan surface, and receives an ultrasonic echo caused due to the ultrasonic beam. Taking a type using piezoelectric elements as an example, the ultrasonic transducer device includes a plurality of ultrasonic transducer elements (ultrasonic element array) and a substrate having a plurality of openings in an array. Also, ultrasonic transducer elements having a monomorphic (unimorphic) structure in which thin piezoelectric elements and a metal plate (vibrating film) are adhered to each other are used. The ultrasonic transducer elements (vibrating elements) are configured to convert electrical vibration into mechanical vibration, and are warped in this case because the size of the metal plate (vibrating film) to which they are adhered is constant even when the piezoelectric elements extend and shrink on the surface.

Furthermore, the ultrasonic transducer device may have a configuration in which several ultrasonic transducer elements arranged in the neighborhood constitute one channel, and a plurality of channels are driven at once to sequentially shift the ultrasonic beam.

Note that a transducer of a type using piezoelectric elements (thin film piezoelectric elements) may be employed as the ultrasonic transducer device, but the present embodiment is not limited to this. For example, a transducer such as c-MUT (Capacitive Micro-machined Ultrasonic Transducers) of a type using capacitive elements, or a bulk-type transducer may be employed. More detailed descriptions of the ultrasonic transducer elements and the ultrasonic transducer device will be given later.

Then, the transmission processing unit 110 performs processing for transmitting an ultrasonic wave to the subject. Furthermore, the transmission processing unit 110 shown in, for example, FIG. 6 includes a transmission pulse generating device 111 and a transmission delaying circuit 113.

Specifically, the transmission pulse generating device 111 applies a transmission pulse voltage to drive the ultrasonic prove 200.

Furthermore, the transmission delaying circuit 113 performs focusing of a transmission wave beam. Accordingly, the transmission delaying circuit 113 gives a time lag between channels with respect to a timing at which the transmission pulse voltage is applied, and converges ultrasonic waves generated by a plurality of vibrating elements. In this manner, by changing a delay time, it is possible to arbitrary change the focal length.

Furthermore, the transmission/reception switch 140 performs processing for switching transmission and reception of an ultrasonic wave. The transmission/reception switch 140 protects a receiving circuit so that no amplitude pulse at the time of transmission is input, and causes a signal at the time of reception to pass through the receiving circuit.

On the other hand, the reception processing unit 120 performs processing for receiving an ultrasonic echo with respect to the transmitted ultrasonic wave. Furthermore, the reception processing unit 120 shown in, for example, FIG. 6 includes a reception delaying circuit 121, a filter circuit 123, and a memory 125.

The reception delaying circuit 121 performs focusing of received wave beams. Because waves reflected on a reflector propagate spherically, the reception delaying circuit 121 gives a delay time so that the waves reach transducers at the same time, and performs addition of the reflective waves taking into consideration the delay time.

Also, the filter circuit 123 performs filter processing using a band-pass filter on the received signal, so as to remove disturbing noise.

Furthermore, the memory 125 stores a received signal output from the filter circuit 123, and the functions thereof can be realized by a memory such as a RAM, an HDD, or the like.

Furthermore, the processing unit 130 performs processing on a received signal from the reception processing unit 120. The processing unit 130 shown in, for example, FIG. 6 includes a scatterer distribution estimation unit 134, a logarithmic conversion processing unit 135, a gain/dynamic range adjustment unit 137, and a sensitivity time control (STC) 139.

Specifically, the scatterer distribution estimation unit 134 performs processing for specifying the reflection intensities from point scatterers in the subject with respect to a received signal, and estimates a point scatterer distribution in the subject. The functions of the scatterer distribution estimation unit 134 will be described in detail later.

The logarithmic conversion processing unit 135 performs log compression, and converts the expression form so that it is easy to recognize the highest and lowest portions of the signal intensity of the received signal at the same time.

Then, the gain/dynamic range adjustment unit 137 adjusts the signal intensity and an area of interest. Specifically, in gain adjustment processing, a direct-current component is added to an input signal subjected to the log compression. Furthermore, in dynamic range adjustment processing, an input signal subjected to the log compression is multiplied by an arbitrary number.

Furthermore, the STC 139 corrects the amplification degree (brightness) depending on the depth, and obtains an image having a uniform brightness on the entire image screen.

Note that the functions of the processing unit 130 can be realized by various types of processors (such as a CPU), hardware such as an ASIC (such as a gate array), programs, and the like.

Also, the DSC 150 performs scan conversion processing on B-mode image data. For example, the DSC 150 converts a line signal into an image signal using interpolation processing such as bilinear interpolation.

Furthermore, the control circuit 160 controls the transmission pulse generating device 111, the transmission delaying circuit 113, the reception delaying circuit 121, the transmission/reception switch 140, and the scatterer distribution estimation unit 134.

Furthermore, the display unit 300 displays image data for display that is generated based on the reconstructed signal. The display unit 300 can be realized by, for example, a liquid crystal display, an organic EL display, an electric paper, or the like.

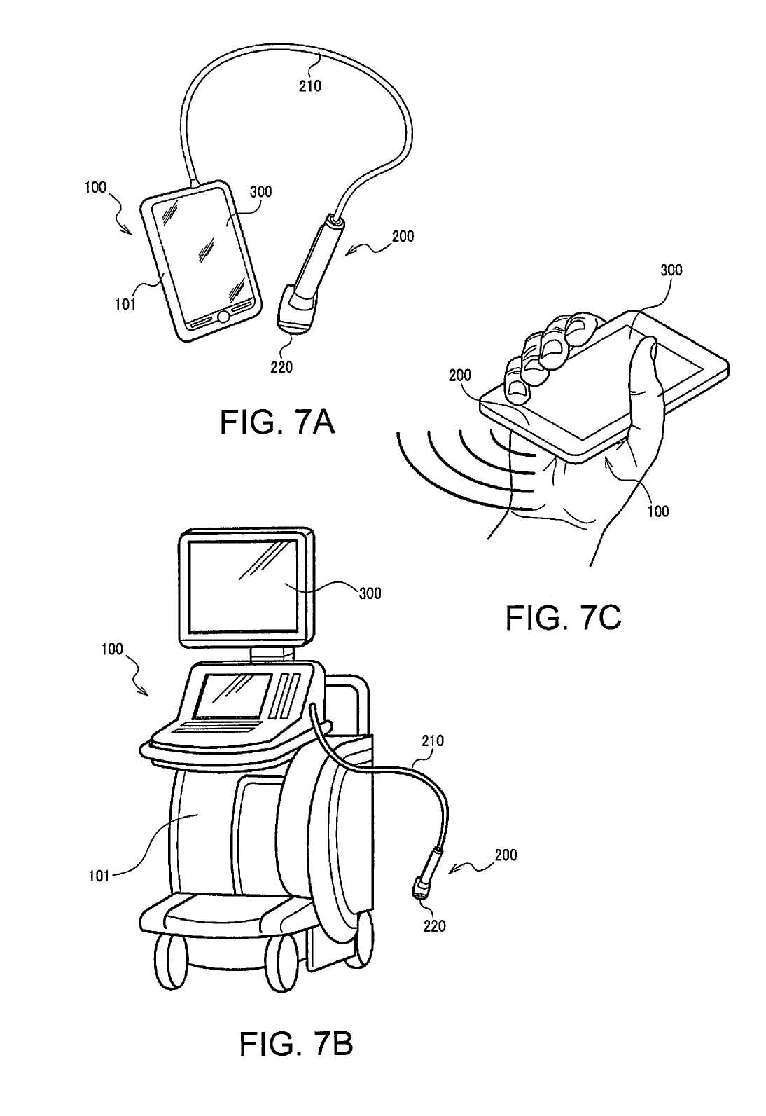

Here, specific examples of device configurations of the ultrasonic imaging device (in a broad sense, electronic device) according to the present embodiment are shown in FIGS. 7A to 7C. FIG. 7A illustrates an example of a handy-type ultrasonic imaging device, and FIG. 7B illustrates an example of a stationary-type ultrasonic imaging device. FIG. 7C illustrates an example of an integral-type ultrasonic imaging device that includes, in its main body, an ultrasonic probe 200.

The ultrasonic imaging devices of FIG. 7A and FIG. 7B include an ultrasonic probe 200 and an ultrasonic measurement device 100, the ultrasonic probe 200 and the ultrasonic measurement device 100 being connected to each other via a cable 210. A probe head 220 is provided at the head of the ultrasonic probe 200, and a main body 101 of the ultrasonic measurement device is provided with the display unit 300 that displays images. In FIG. 7C, the ultrasonic probe 200 is provided in the ultrasonic imaging device having the display unit 300. The ultrasonic imaging device of FIG. 7C can be realized by a general-purpose mobile information terminal such as a Smartphone, for example.

2.2. Detail of Processing

2.2.1. Reflection Intensity Specifying Processing

The processing unit 130 of the present embodiment performs, on a received signal, processing for specifying the reflection intensities from point scatterers in the subject based on M basis waves (where M is an integer of 2 or more) at least two of which have phases shifted from each other with a phase difference smaller than the phase difference corresponding to the pulse width of a transmission pulse signal transmitted from the transmission processing unit 110 or the pulse width of a received signal in response to the transmission pulse signal.

In the present embodiment, as shown in, for example, FIG. 8A, an ultrasonic pulse signal TPS having a wavelength λ is transmitted to the subject. It is assumed that the subject of this example includes, for example, three point scatterers (SP1 to SP3). The point scatterer SP1 and the point scatterer SP2 are separated from each other by λ/2, and the point scatterer SP2 and the point scatterer SP3 are separated from each other by λ/4. Note that for ease of description, the number of the point scatterers included in the subject is set to three, but actually, the larger number of point scatterers are included.

It is assumed that a wave received by the reception processing unit 120 as a result of transmission of the pulse signal TPS by the transmission processing unit 110 to the subject is the received wave X as shown in FIG. 8B. In this example, the received wave X is decomposed into basis waves (basis functions) sᵢ as shown in FIG. 8C, specifies the reflection intensity aᵢ from the point scatterer in the subject, and specifies the scatterer density distribution in the subject. Specifically, the reflection intensity from the point scatterer SP1 is 0.5, the reflection intensity from the point scatterer SP2 is 1.0, and the reflection intensity from the point scatterer SP3 is 0.7.

Here, in a case where, for example, M basis waves are provided, a phase difference between the i-th basis wave sᵢ and the (i+1)-th basis wave sᵢ₊₁ of the M basis waves is smaller than a phase distance that corresponds to the pulse width of a transmission pulse signal or the pulse width of the received signal. Note that M is an integer of 2 or more, and i is an integer of 1≦i≦M.

Accordingly, the received wave X with which backscattered waves from a plurality of scatterers interferes is decomposed into basis waves (s₀ to s_{M}) before the interference that have changed phases, and thus it is possible, for example, to provide a high quality image that has a distance resolution higher than that determined depending on the ultrasonic pulse width.

According to the present embodiment, it is thus possible to improve not only the azimuth resolution but also the distance resolution of a subject measurement result using an ultrasonic wave.

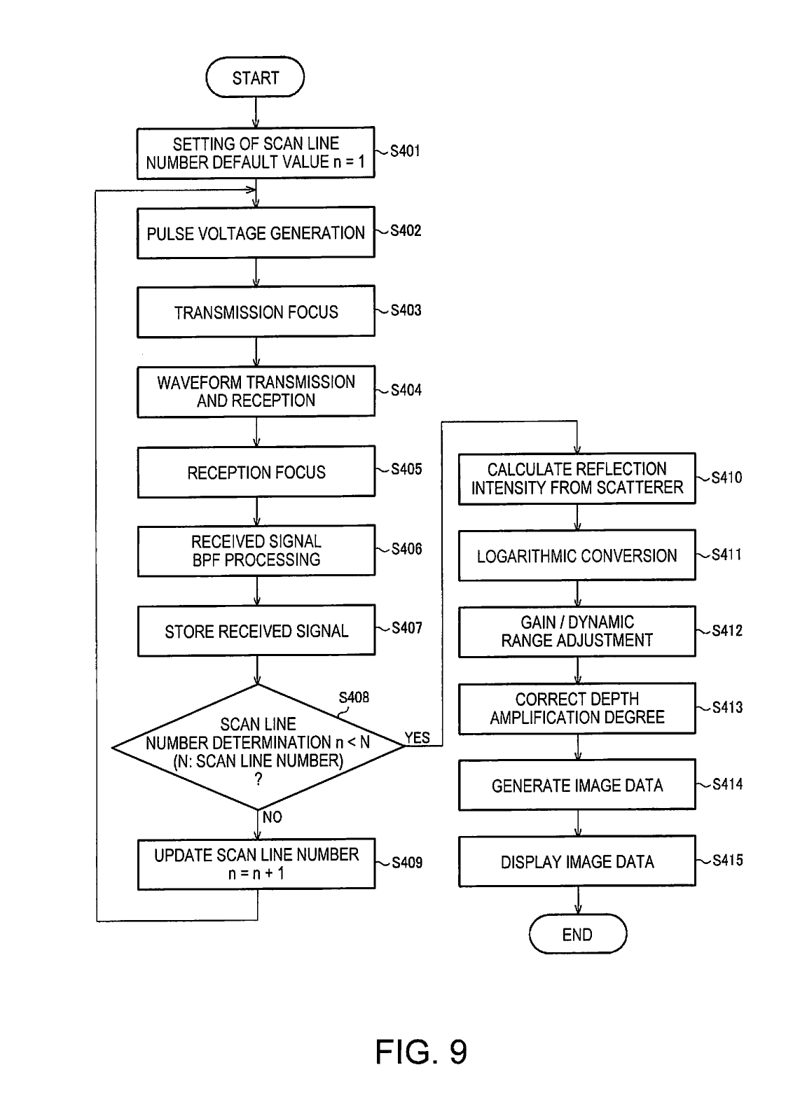

Hereinafter, the flow of specific processing of the present embodiment will be described with reference to the flowchart of FIG. 9.

First, a default value of a scan line number n is set to 1 (S401).

Then, the transmission pulse generating device 111 generates a pulse voltage (S402).

Then, the transmission delaying circuit 113 performs transmission focus control (S403), and the ultrasonic prove 200 emits an ultrasonic beam corresponding to the generated pulse voltage to a subject (S404). Furthermore, the ultrasonic prove 200 receives an ultrasonic echo obtained by the emitted ultrasonic beam being reflected on and returning from the subject (S404).

The reception delaying circuit 121 performs reception focus control on this ultrasonic echo (S405), the filter circuit 123 performs band-pass filter (BPF) processing on the received signal subjected to the reception focus control (S406), and the memory 125 stores the received signal subjected to the BPF processing (S407).

Then, it is determined whether or not all the scan lines have been subjected to the processing of steps S402 to S407 (S408). Specifically, it is determined whether or not the current scan line number n is smaller than the total scan line number N.

If it is determined that all the scan lines have not been subjected to the processing of steps S402 to S407, that is, if it is determined that the current scan line number n is smaller than the total scan line number N, the current scan line number n is incremented by 1 (S409), and the processing of steps S402 to S408 is performed again.

On the other hand, if it is determined, in step S408, that all the scan lines have been subjected to the processing of steps S402 to S407, that is, if it is determined that the current scan line number n is equal to the total scan line number N, the scatterer distribution estimation unit 134 performs processing for specifying the reflection intensities from point scatterers in the subject on the received signal (S410).

Here, a specific example of the processing for specifying the reflection intensity will be described. First, in this example, the ultrasonic measurement device 100 of the present embodiment is assumed to include a storage unit in which respective pieces of basis wave data on M basis waves are stored in association with M measurement points set in the depth direction of the subject.

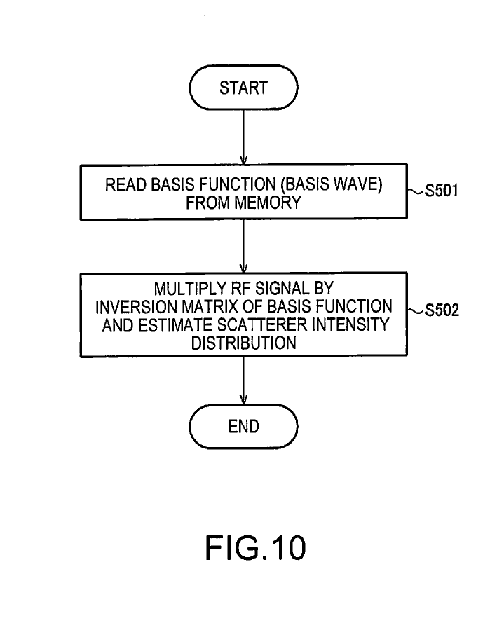

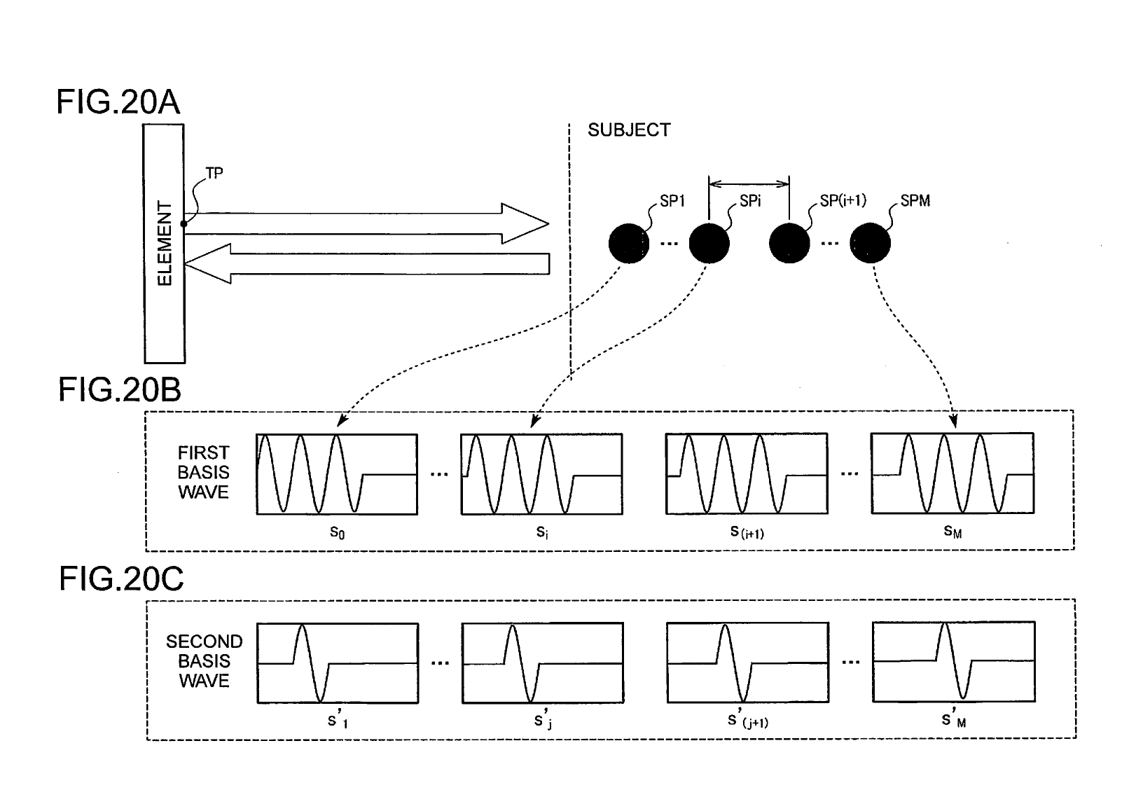

Accordingly, as shown in the flowchart of FIG. 10, it is possible for the scatterer distribution estimation unit 134 to read the respective pieces of basis wave data of M basis waves from the storage unit (memory) (S501), and to decompose the received wave into M basis wave components, for example (S502). The same will apply to a first basis wave of a second embodiment, which will be described later with reference to FIGS. 20A and 20B.

Specifically, in step S502, the scatterer distribution estimation unit 134 estimates a scatterer intensity distribution by multiplying a RF signal (received signal) by the inversion matrix of the basis function (S502). That is, the processing unit 130 performs deconvolution processing of the M basis waves on the received signal and thereby obtains the reflection intensities.

Accordingly, it is possible, for example, to specify the positions of the scatterers in the subject based on the reflection intensities.

Then, the logarithmic conversion processing unit 135 performs logarithmic conversion processing of the specified reflection intensities (S411).

Also, the gain/dynamic range adjustment unit 137 adjusts the signal intensity and an area of interest (S412), and the STC 139 corrects the amplification degree (brightness) depending on the depth (S413).

Furthermore, the DSC 150 performs scan conversion processing to generate B-mode image data (image data for display) (S414), the display unit 300 displays the generated image data for display (S415), and the processing ends.

Accordingly, in this example, the processing unit 130 performs processing for generating a B-mode image based on the reflection intensities specified using the specifying processing.

Accordingly, it is possible, for example, to display, on the display unit 300, an image that makes a user easily understandable the state of the inside of the subject.

2.2.2. Basis Wave Generation Processing

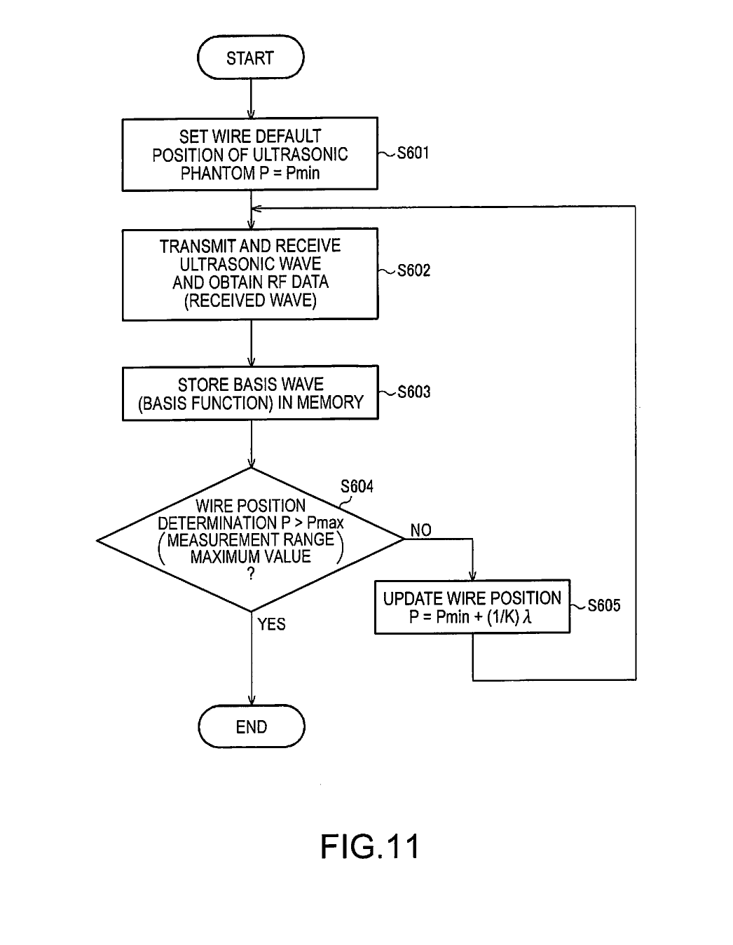

Meanwhile, the basis wave used in the processing of the flowcharts of FIGS. 9 and 10 needs to be generated in advance before the processing is performed (in preprocessing), and to be stored in a memory (not shown). In the following, the flow of the processing for generating a basis wave will be described with reference to the flowchart of FIG. 11, and to 12A to 12C.

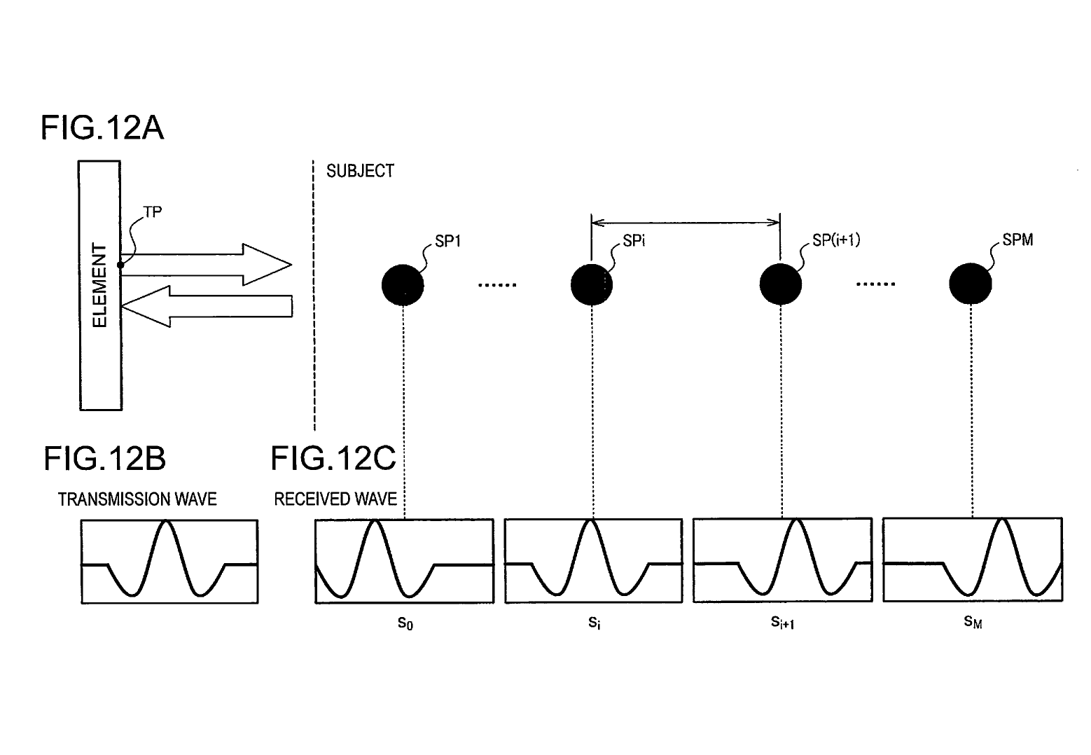

First, a wire default position P of an ultrasonic phantom is set (S601). Here, the default position P is set at a position Pₘᵢₙ that is closest to the measurement surface of the ultrasonic prove of the ultrasonic measurement device. In the example of FIG. 12A, Pₘᵢₙ is a position at which the point scatterer SP1 is positioned.

Then, the transmission processing unit 110 transmits an ultrasonic pulse signal as shown in FIG. 12B to a subject (ultrasonic phantom), and the reception processing unit 120 receives a signal (RF data) that corresponds to the transmission pulse signal (S602). When, for example, the wire position P is located at the position of the point scatterer SPi, a received wave sᵢ as shown in FIG. 12C can be received. In the present example, the processing unit 130 stores, in the memory, the received wave that was received directly as a basis wave (S603).

Then, the processing unit 130 determines whether or not the wire position P is a value larger than the maximum value Pₘₐₓ of the measurement range (S604), and if it is determined that the wire position P is a value equal to or smaller than the maximum value Pₘₐₓ of the measurement range, the wire position P is updated based on the above-described formula (4) (S605), and the procedure returns to step S602. In step S603 thereafter, a received wave sᵢ₊₁ is received, and is stored as a basis wave in the memory.

On the other hand, if it is determined that the wire position P is a value larger than the maximum value Pₘₐₓ of the measurement range, the processing unit 130 ends the procedure.

In summary, as shown in, for example, FIGS. 12A to 12C, the i-th basis wave sᵢ of M basis waves has a waveform that corresponds to the ultrasonic signal received from the i-th point scatterer SPi arranged at the i-th measurement point. Note that i is an integer of 1≦i≦M.

Then, the (i+1)-th basis wave s₍ᵢ₊₁₎ of M basis waves is the waveform that corresponds to the received ultrasonic signal from the (i+1)-th point scatterer SP(i+1) arranged at the (i+1)-th measurement point, which is farther away from the transmission point TP of the ultrasonic wave than the i-th measurement point.

Accordingly, it is possible, for example, to extract basis wave components from the received wave with the distance resolution that corresponds to the set distance between the measurement points.

However, the basis wave generation method of the present embodiment is not limited to the above-described method. For example, each basis wave of M basis waves may be a waveform obtained by shifting the phase of the pulse wave of a transmission pulse signal.

That is, in the above-described example, a received wave in response to a transmission wave is set as a basis wave, but in a modification thereof, when a basis wave is generated, instead of the ultrasonic wave transmission/reception processing being performed, the phase of the transmission wave is shifted so that a basis wave as shown in FIG. 12C is generated.

Accordingly, it is possible, for example, to generate a basis wave without performing the ultrasonic wave transmission/reception processing.

Furthermore, in order to improve the distance resolution, it is sufficient that a set distance between measurement points is made shorter and the number of basis waves is increased, but an increase in the number of basis waves will need a larger storage capacity for storing the basis waves, causing the problem that the processing load is also large.

In contrast, in the actual measurement scene, there is often the case where the distance resolution is desired to be improved in not all depth ranges of a subject but only an arbitrary depth range.

Therefore, in the present embodiment, a set distance between measurement points in a first depth range of the subject may be smaller than a set distance between measurement points in a second depth range of the subject. That is, in the subject, the set distance between measurement points is set to be shorter in the depth range in which measurement with an accurate distance resolution is desired, and the set distance between measurement points is set to be greater in the depth range in which measurement with a rough distance resolution is desired.

Accordingly, in the subject, it is possible, for example, to reduce the number of basis waves while improving the distance resolution in the first depth range. As a result, it is possible to suppress the storage capacity needed for storing the basis waves, and also to reduce the processing load of the ultrasonic measurement device 100.

Furthermore, in the extreme case, it is also possible that the processing unit 130 performs the reflection intensity specifying processing in the first depth range of the subject, and does not perform the reflection intensity specifying processing in the second depth range of the subject.

Accordingly, it is possible, for example, to generate a B-mode image in which only the depth range of the subject that is needed to be measured is imaged, and to reduce the processing load of the ultrasonic measurement device 100.

2.2.3. Measurement Result

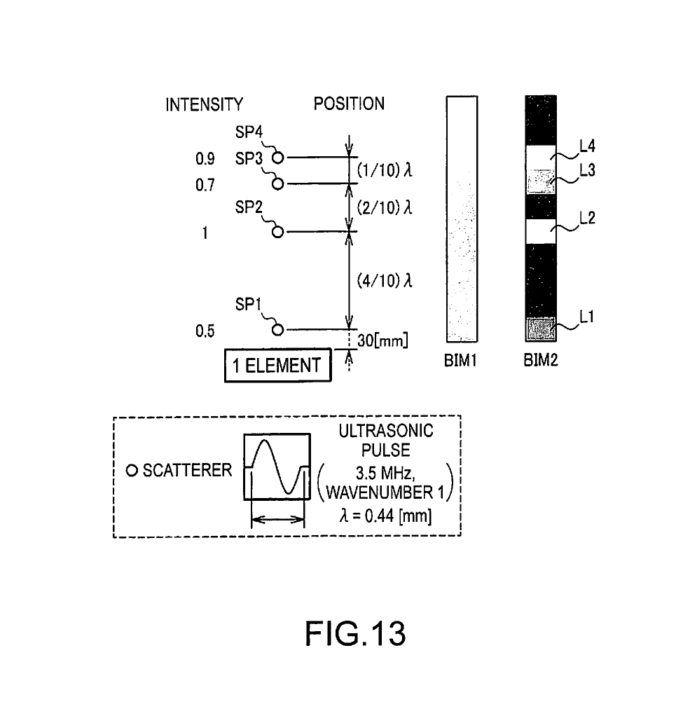

Hereinafter, FIG. 13 shows an example of a measurement result according to the present embodiment. In the example, as shown in FIG. 13, an ultrasonic wave pulse having the wavelength λ of 0.44 mm, the wavenumber of 1, the frequency of 3.5 MHz is transmitted to the subject.

In the example of FIG. 13, it is assumed that the upward direction of the drawing is the depth direction of the subject, and there are four point scatterers (SP1 to SP4) in the subject. Also, it is assumed that the ultrasonic transducer element and the point scatterer SP1 are separated from each other by 30 mm, the point scatterer SP1 and the point scatterer SP2 are separated from each other by (4/10)λ, the point scatterer SP2 and the point scatterer SP3 are separated from each other by (2/10)λ, and the point scatterer SP3 and the point scatterer SP4 are separated from each other by (1/10)λ. Furthermore, it is assumed that the reflection intensity from the point scatterer SP1 is 0.5, the reflection intensity from the point scatterer SP2 is 1.0, the reflection intensity from the point scatterer SP3 is 0.7, and the reflection intensity from the point scatterer SP4 is 0.9.

In this case, when a B-mode image is generated only using the phase inversion method and the filter method, an image BIM1 in the same figure is generated. In this image BIM1, because reflective waves from the point scatterers overlap each other and the tone of the entire image hardly changes, it is difficult to specify the detailed positions of the four point scatterers.

On the other hand, when a B-mode image is generated based on the reflection intensities specified by the above-described embodiment, an image BIM2 of the same figure is generated. It is clear from this image BIM2 that a color layer L1 corresponds to the reflection from the point scatterer SP1, a color layer L2 corresponds to the reflection from the point scatterer SP2, a color layer L3 corresponds to the reflection from the point scatterer SP3, and a color layer L4 corresponds to the reflection from the point scatterer SP4. That is, the distance resolution is improved as compared with the case using only the phase inversion method and the filter method.

### 3. Second Embodiment

3.1. System Configuration Example

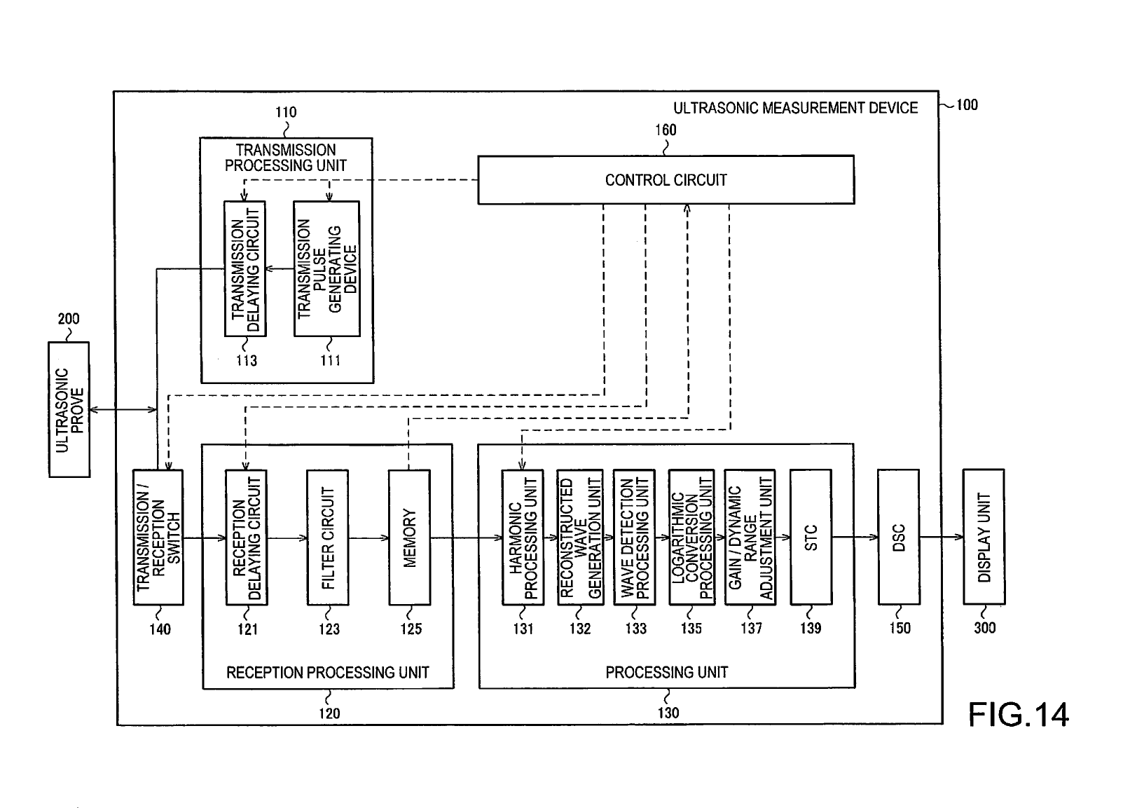

Hereinafter, an example of a specific configuration of an ultrasonic imaging device according to a second embodiment will be shown in FIG. 14. Similarly to the first embodiment of FIG. 6, the ultrasonic imaging device includes the ultrasonic measurement device 100, the ultrasonic prove 200, and the display unit 300. Furthermore, the ultrasonic measurement device 100 of the present embodiment includes the transmission processing unit 110, the reception processing unit 120, the processing unit 130, the transmission/reception switch 140, the DSC 150, and the control circuit 160. The configurations and functions of the transmission processing unit 110, the reception processing unit 120, the transmission/reception switch 140, the DSC 150, the control circuit 160, the ultrasonic prove 200, and the display unit 300 are the same as those of the first embodiment described with reference to FIG. 6, and thus descriptions thereof are omitted.

The ultrasonic measurement device 100 of the present embodiment differs from the ultrasonic measurement device 100 of the above-described first embodiment in the configuration of the processing unit 130. That is, the processing unit 130 of the present embodiment includes a harmonic processing unit 131, a reconstructed wave generation unit 132, a wave detection processing unit 133, the logarithmic conversion processing unit 135, the gain/dynamic range adjustment unit 137, and the STC 139. Note that the functions of the logarithmic conversion processing unit 135, the gain/dynamic range adjustment unit 137, and the STC 139 are the same as those of the above-described example, and thus descriptions thereof are omitted.

Specifically, as described above, the harmonic processing unit 131 performs processing for extracting a harmonic component (mainly a third harmonic component).

As will be described in detail later, the reconstructed wave generation unit 132 performs processing for converting a received signal into a reconstructed signal based on the extracted harmonic component (mainly the third harmonic component).

Then, the wave detection processing unit 133 performs absolute value (rectifying) processing and then extracts an unmodulated signal using a low-pass filter.

Note that the ultrasonic measurement device 100 and the ultrasonic imaging device including this are not limited to the configuration of FIG. 14, and various modifications, such as one in which some constituent components of the devices are omitted or one in which another constituent component is added, can be executed. Furthermore, some or all of the functions of the ultrasonic measurement device 100 of the present embodiment and the ultrasonic imaging device including this may be realized by a server connected via communication.

3.2. Detail of Processing

3.2.1. Reconstructed Wave Generation Processing

The processing unit 130 of the present embodiment subjects the received signal in response to a transmission pulse signal transmitted by the transmission processing unit 110 to processing for specifying binding coefficients for a plurality of first basis waves constituting the received signal, and performs processing for converting the received signal into a reconstructed signal, based on the plurality of binding coefficients specified by the binding coefficient specifying processing and a second basis wave whose wavenumber is smaller than that of a first basis wave.

Accordingly, it is possible to measure the inside of the subject based on the generated reconstructed signal. This reconstructed signal is constituted by the second basis wave whose wavenumber is smaller than that of the first basis wave.

Here, the reconstructed signal (reconstructed wave) refers to a signal that is obtained in a manner that reflected signal components (reflective wave components) that are reflected from point scatterers in the subject and are included in the received signals (received waves) are replaced by signals (waveforms) having a pulse width smaller than that of the original reflected signal components, and the replaced signals (waveforms) are overlapped again at the same timing as that of the timing of receiving original reflected signal components. The reconstructed signal refers to, for example, a reconstructed wave as shown in FIG. 15D, which will be described later. That is, the above-described first basis waves respectively correspond to the reflected signal components (reflective wave components) from the point scatterers, and the second basis waves respectively correspond to the signals (waveforms) that are replaced by the first basis waves and whose pulse widths are smaller than that of the original reflected signal components.

Also, each first basis wave is a wave that corresponds to the reflective wave component of the received signal from a point scatterer located in a predetermined depth in the subject, and that is shown in, for example, FIG. 15B, which will be described later. As described later, by determining whether or not the received signal includes a first basis wave component, it is possible to determine whether or not there is the point scatterer that corresponds to this first basis wave in the subject. Furthermore, if it is possible to determine that the point scatterer corresponding to this first basis wave is included in the subject, reflection characteristics and the like of the point scatterer can be specified based on the signal intensity (reflection intensity) of the first basis wave component included in the received signal. This processing for specifying the reflection intensity is the binding coefficient specifying processing. Note that the second basis wave will be described later.

Accordingly, in the present embodiment, since the reflective wave components of the received wave are replaced by the second basis waves whose pulse widths are short, and the received waves are reconstructed, it is possible to improve the distance resolution of the measurement result of the subject based on the reconstructed signal. Accordingly, it is possible to improve not only the azimuth resolution but also the distance resolution of the measurement result of the subject due to an ultrasonic wave.

Specifically, the processing unit 130 subjects the harmonic (received wave X) corresponding to the received signal as shown in, for example, FIG. 15A to processing for specifying the binding coefficients for the first basis waves (Sᵢ) as shown in FIG. 15B, and then to processing for converting the first basis waves (Sᵢ) constituting the received wave X into the second basis waves (S′ᵢ) as shown in FIG. 15C, so as to generate the reconstructed wave X′ serving as the reconstructed signal as shown in FIG. 15D, based on the second basis waves (S′ᵢ). Note that the harmonic X shown in FIG. 15A may be a harmonic that is extracted by performing filter processing on the original received wave, or the like.

Accordingly, it is possible, for example, to convert the received wave into the reconstructed wave that is constituted by the second basis waves whose wavenumber is smaller than that of the first basis waves, and to improve the distance resolution of a measurement result of the subject.

Here, the first basis waves are harmonics that can be extracted from the received signal. Accordingly, it is possible to decompose the received signal into a plurality of first basis waves.

In the example of FIG. 15B for example, the first basis waves are waves indicated by the basis function Sᵢ. In the present embodiment, there is not one first basis wave but a plurality of waves. In the example of FIG. 15B for example, the plurality of first basis waves are M first basis waves, where M is an integer of 2 or more, and i is an integer of 1≦i≦M.

Of the M first basis waves, the i-th first basis wave has a phase difference that is shorter than the phase difference that corresponds to the pulse width of the transmission pulse signal or the pulse width of the received signal, and has a phase shifted from that of the (i+1)-th first basis wave.

Accordingly, it is possible, for example, to measure a subject with the distance resolution of the distance that is shorter than the phase difference corresponding to the pulse width of the transmission pulse signal or the pulse width of the received signal.

Furthermore, when processing for specifying the binding coefficients for a plurality of first basis waves constituting the received wave X is performed, as shown in FIG. 15B, a binding coefficient aᵢ that corresponds to each first basis wave Sᵢ is obtained. This binding coefficient aᵢ is a value that is determined how much extent the corresponding first basis wave Sᵢ is included in the received wave X. That is, the received wave X is, as shown in the formula (2) below, indicated by the sum of the products of the first basis wave and the binding coefficient.

$$\begin{matrix}[ Formula 2 ] & \\ X = {\sum }_{i = 1}^{M} ( {s}_{i} \cdot {a}_{i} ) & ( 2 )\end{matrix}$$

Furthermore, the second basis waves are waves that have the same phase difference as that of the first basis waves and whose wavenumber is smaller than that of the first basis waves. Note that this phase difference is a phase difference between the i-th first basis wave and the (i+1)-th first basis wave.

Also, each second basis wave S′ᵢ is associated with the binding coefficient aᵢ that is obtained when the received wave X is decomposed into a plurality of first basis waves Sᵢ (binding coefficient specifying processing). Accordingly, the received wave X is indicated by the formula (3) below.

$$\begin{matrix}[ Formula 3 ] & \\ {X}^{′} = {\sum }_{i = 1}^{M} ( {s}_{i}^{′} \cdot {a}_{i}^{′} ) & ( 3 )\end{matrix}$$

Accordingly, it is possible, for example, to make the pulse width of the basis waves constituting the received wave short.

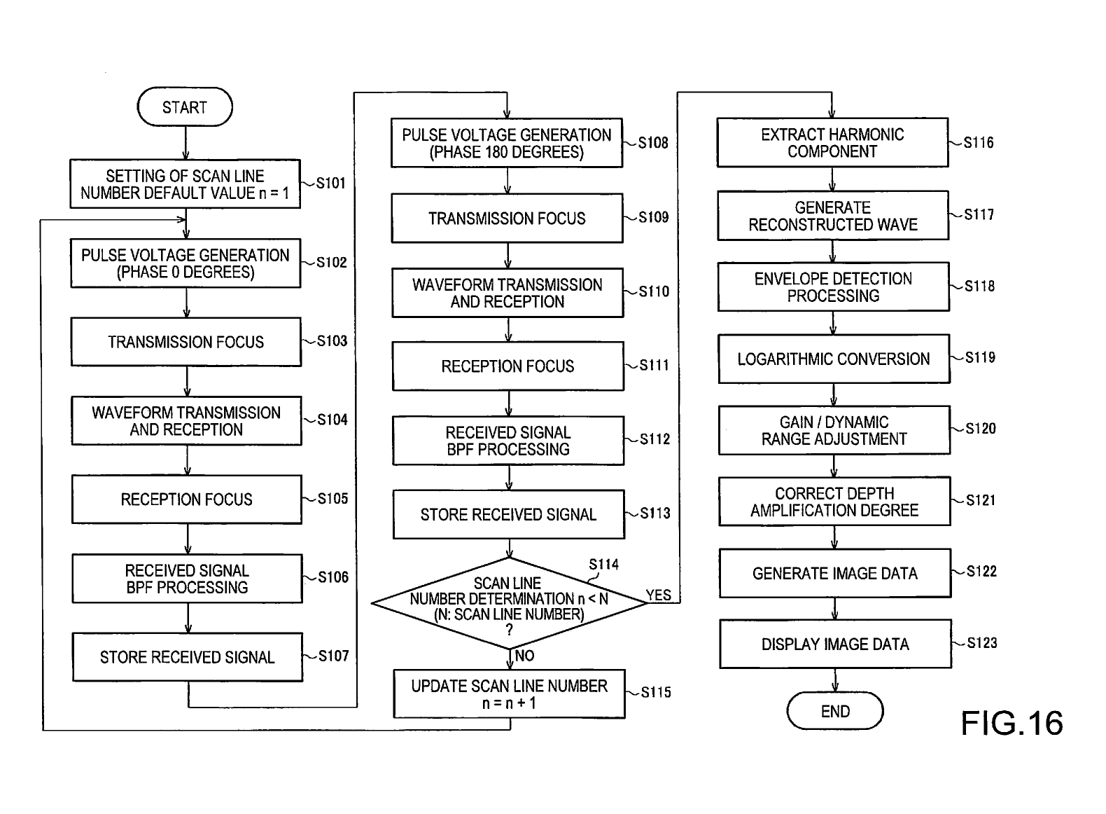

Hereinafter, the flow of the processing according to the present embodiment will be described with reference to the flowchart of FIG. 16.

First, the default value of the scan line number n is set to 1 (S101).

Then, the transmission pulse generating device 111 generates a pulse voltage having a phase of 0 degrees (S102).

Then, the transmission delaying circuit 113 performs transmission focus control (S103), and the ultrasonic prove 200 emits an ultrasonic beam that corresponds to the generated pulse voltage to the subject (S104). Furthermore, the ultrasonic prove 200 receives an ultrasonic echo that is obtained by the emitted ultrasonic beam being reflected on and returning from the subject (S104).

In contrast, the reception delaying circuit 121 performs reception focus control (S105), and the filter circuit 123 performs band-pass filter (BPF) processing on the received signal after the reception focus control (S106), and stores the received signal subjected to the BPF processing in the memory 125 (S107).

Then, the transmission pulse generating device 111 generates a pulse voltage of a phase of 180 degrees (S108). Then, the phase-inverted pulse is subjected to the same processing as the processing of the above-described steps S102 to S107 (S109 to S113).

Thereafter, it is determined whether or not the processing of steps S102 to S113 has been performed on all the scan lines (S114). Specifically, it is determined whether or not the current scan line number n is smaller than the total scan line number N.

If it is determined that the processing of steps S102 to S113 has not been performed on all the scan lines, that is, the current scan line number n is smaller than the total scan line number N, the current scan line number n is incremented by 1 (S115), and the processing of steps S102 to S114 is performed again.

On the other hand, if it is determined in step S115 that processing of steps S102 to S113 has been performed on all the scan lines, that is, the current scan line number n is equal to the total scan line number N, the harmonic processing unit 131 performs processing for extracting a harmonic component (harmonic component) (S116). Specifically, in the present extracting processing, as shown in FIGS. 2A to 2C above, subtracting processing with respect to the received wave corresponding to the transmission wave of a phase of 0 degrees and the received wave corresponding to the transmission wave of a phase of 180 degrees is performed so as to extract the fundamental and a third harmonic. Thereafter, as shown in FIGS. 3A and 3B, the extracted fundamentals and third harmonic are subjected to high-pass filter processing to extract only the third harmonic.

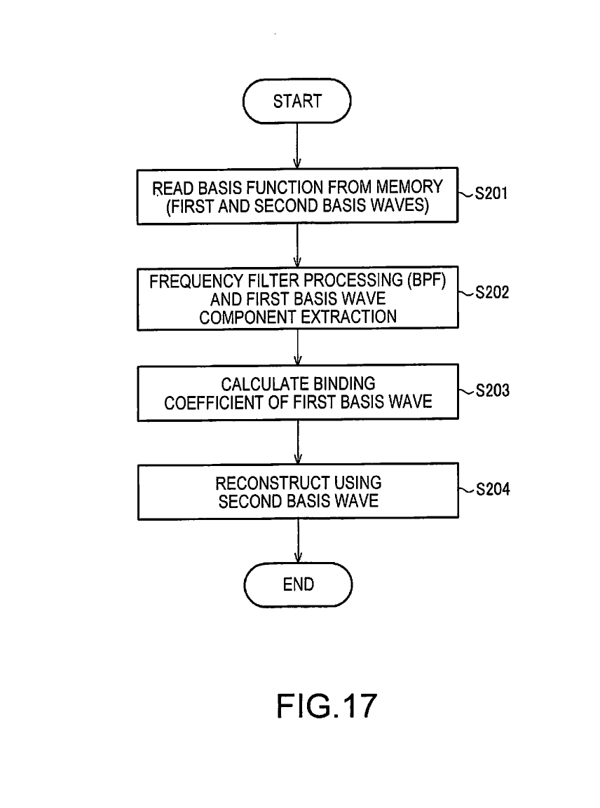

Then, the reconstructed wave generation unit 132 generates a reconstructed wave based on the extracted third harmonic (S117). Here, the flow of the processing for generating a reconstructed wave according to the present embodiment is shown in the flowchart of FIG. 17.

First, the reconstructed wave generation unit 132 reads the basis functions (the first basis waves and the second basis waves) from a memory (not shown) (S201). Then, the reconstructed wave generation unit 132 performs frequency filter processing (BPF), and extracts first basis wave components of the third harmonic based on the first basis waves read from the memory, as shown in FIG. 15B above (S202).

Then, the reconstructed wave generation unit 132 performs processing for specifying the binding coefficients for the first basis waves constituting the third harmonic based on the extraction result (S203). The processing for specifying binding coefficients is processing for specifying the above-described reflection intensities. In the processing for specifying binding coefficients, the reflection characteristics and the like of the point scatterers are specified, based on the signal intensities (reflection intensities) of the first basis wave components included in the received signal.

Specifically, the reconstructed wave generation unit 132 (processing unit 130) performs, as the processing for specifying binding coefficients, deconvolution processing of the received signal.

Accordingly, it is possible, for example, to specify the binding coefficients of the first basis waves constituting the received wave.

Then, the reconstructed wave generation unit 132 replaces the respective first basis waves by the second basis waves having the same phase and generates a reconstructed wave based on the specified binding coefficients and second basis waves (S204). In other words, processing for converting a received signal into a reconstructed signal is performed.

Specifically, the processing unit 130 performs convolution processing of the second basis waves, the convolution processing serving as the reconstructed signal conversion processing.

Accordingly, it is possible, for example, to generate a reconstructed wave based on the second basis waves whose wavenumber is reduced relative to that of the first basis waves, which constitute the received wave or a harmonic extractable from the received wave.

Then, the wave detection processing unit 133 performs absolute value (rectifying) processing on the generated reconstructed wave, and then extracts an unmodulated signal using a low-pass filter (S118), and the logarithmic conversion processing unit 135 performs logarithmic conversion processing (S119).

Then, the gain/dynamic range adjustment unit 137 adjusts the signal intensity and an area of interest (S120), and the STC 139 corrects the amplification degree (brightness) depending on the depth (S121).

Furthermore, the DSC 150 performs scan conversion processing so as to generate B-mode image data (image data for display) (S122), the display unit 300 displays the generated image data for display (S123), and the procedure ends.

3.2.2. First and Second Basis Wave Generation Processing

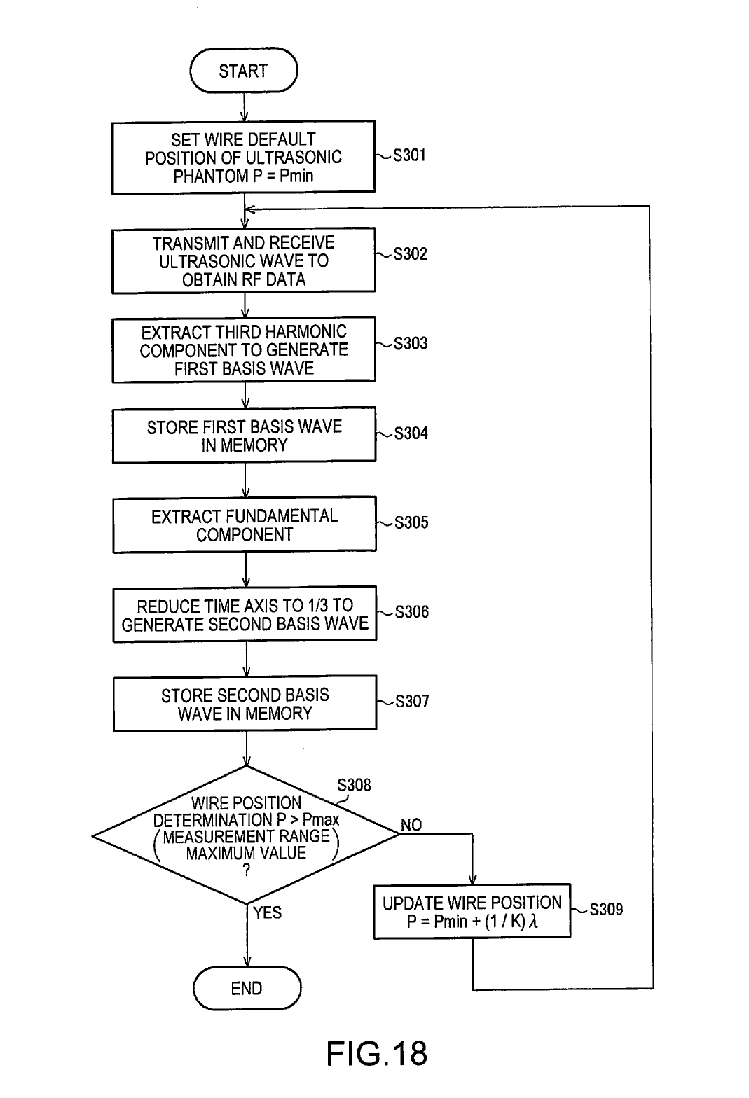

The first basis waves and the second basis waves used in the processing of the flowchart of FIGS. 16 and 17 need to have been generated (in preprocessing) before performing the processing, and to have been stored in the memory (not shown). Hereinafter, the flow of the first and second basis wave generation processing will be described with reference to the flowchart of FIG. 18.

First, the wire default position P of an ultrasonic phantom is set (S301). Here, the default position P is set to the position Pₘᵢₙ that is closest to the measurement surface of the ultrasonic prove of the ultrasonic measurement device. Furthermore, a wire position refers to the position of a point scatterer.

Then, the transmission processing unit 110 transmits two pulse signals whose phases are inverted from each other to the subject (ultrasonic phantom), and the reception processing unit 120 receives two reception signals (RF data) in response to the two transmission pulse signals (S302).

Then, the processing unit 130 performs, as shown in FIGS. 2B and 2C above, subtracting processing on the two received signals in response to the two transmission pulse signals, so as to obtain one differential signal, and performs, as shown in FIGS. 3A and 3B above, first filter processing on the obtained differential signal to extract a harmonic component (third harmonic component). Here, the first filter processing refers to, for example, high-pass filter processing, band-pass filter processing, or the like.

Furthermore, the processing unit 130 obtains, as a first basis wave, the harmonic that corresponds to the reflective wave component from the point scatterer arranged at a given measurement point, based on the extracted harmonic component (S303).

In the above-described example of FIGS. 15A and 15B, the received wave (harmonic) X includes reflective wave components from various point scatterers in the subject, and thus it is impossible to specify a first basis wave component from the given measurement point that is included in the received wave X, unless comparison with the first basis wave specified in advance is performed. In contrast, in the present processing, the harmonic component after the first filter processing includes only the reflective wave component from the point scatterer arranged at a given measurement point in the ultrasonic phantom. This harmonic component includes noise and the like, but the noise component can easily be separated.

Therefore, it is possible, for example, to specify the first basis wave corresponding to the reflective wave component from the point scatterer at a given measurement point in the subject. The processing unit 130 then stores the specified first basis wave in the memory (S304).

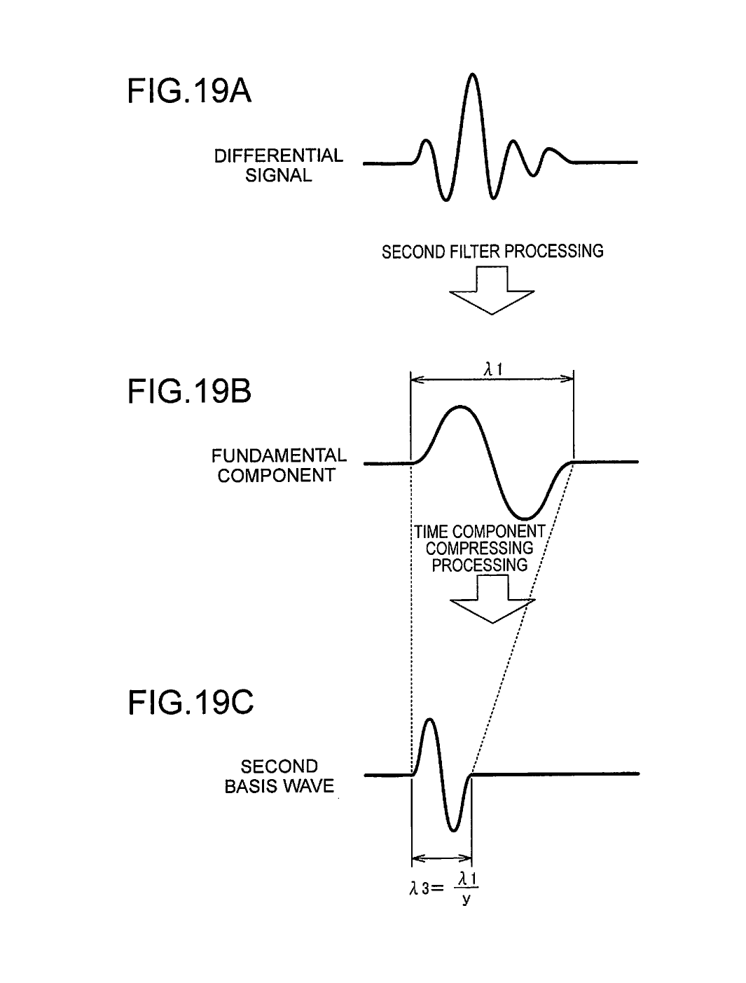

Furthermore, the processing unit 130 performs second filter processing on the differential signal obtained by the subtracting processing shown in FIG. 19A so as to extract a basis wave component as shown in FIG. 19B (S305). Then, based on the extracted fundamental component, the processing unit 130 obtains the fundamental that corresponds to the reflective wave component from the point scatterer arranged at a given measurement point and performs time component compression processing as shown in FIG. 19C on the obtained fundamental to obtain a second basis wave (S306). In a case where processing for compressing the time component to 1/y is performed, a wavelength λ3 of the second basis wave of FIG. 19C is 1/y (where y is a positive value) of the wavelength λ1 of the fundamental of FIG. 19B.

Accordingly, it is possible, for example, to specify the second basis wave whose wavelength is smaller than that of the first basis wave that corresponds to the reflective wave component from the point scatterer arranged at a given measurement point of the subject.

Furthermore, the second basis wave can be obtained by performing time component compression processing on the fundamental that can be extracted from the received signal.

Accordingly, it is possible, for example, to specify the second basis wave, using simple processing such as subtracting processing, filter processing, and time component compression processing. Also, the processing unit 130 stores the specified second basis wave in the memory (S307).

Thereafter, the processing unit 130 determines whether or not the wire position P is a value larger than the maximum value Pₘₐₓ of the measurement range (S308), and if it is determined that the wire position P is a value equal to or smaller than the maximum value Pₘₐₓ of the measurement range, the wire position P is updated based on the formula (4) below (S309), and procedure returns to step S302. Note that in the formula (4), K is a given constant, and λ is a wavelength.

[Formula 4]

P=Pₘᵢₙ+(1/K)λ (4)

On the other hand, if it is determined that the wire position P is a value larger than the maximum value Pₘₐₓ of the measurement range, the processing unit 130 ends the processing.

In summary, as shown in, for example, FIGS. 20A and 20B, the i-th first basis wave sᵢ of the M first basis waves is a harmonic that corresponds to the received ultrasonic signal from the i-th point scatterer SPi arranged at the i-th measurement point. Note that M is an integer of 2 or more, and i is an integer of 1≦i≦M.

Furthermore, the (i+1)-th first basis wave S(i+1) of the M first basis waves is a harmonic that corresponds to the received ultrasonic signal from the (i+1)-th point scatterer SP(i+1) arranged at the (i+1)-th measurement point that is farther away from the transmission point TP of the ultrasonic wave than the i-th measurement point.

Accordingly, it is possible, for example, to extract the first basis wave component from the received wave with the distance resolution corresponding to the set distance between the measurement points.

Also, as shown in FIGS. 20A and 20C for example, the j-th second basis wave s′ⱼ of N second basis waves is a harmonic corresponding to the received ultrasonic signal from the j-th point scatterer SPj arranged at the j-th measurement point. Note that N is an integer of 2 or more, and j is an integer of 1≦j≦N. In the present example, M=N is satisfied, but M≠N may be used.

Furthermore, the (j+1)-th second basis wave s′₍ⱼ₊₁₎ of the N second basis waves is a harmonic that corresponds to the received ultrasonic signal from the (j+1)-th point scatterer SP(j+1) arranged at the (j+1)-th measurement point that is located farther away from the transmission point TP of the ultrasonic wave than the j-th measurement point.

Accordingly, it is possible, for example, to improve the distance resolution of the measurement result of the subject using an ultrasonic wave to the set distance between the measurement points.

However, the first and second basis wave generation processing according to the present embodiment is not limited to the above-described processing. For example, each second basis wave may be generated by reducing the wavenumber of the corresponding first basis wave. Furthermore, instead of performing the first and second basis wave generation processing, the first basis wave and the second basis wave that are stored in advance in the memory may be used. Note that the above-described ultrasonic probe and measurement point may be of simulation.

3.2.3. Measurement Result

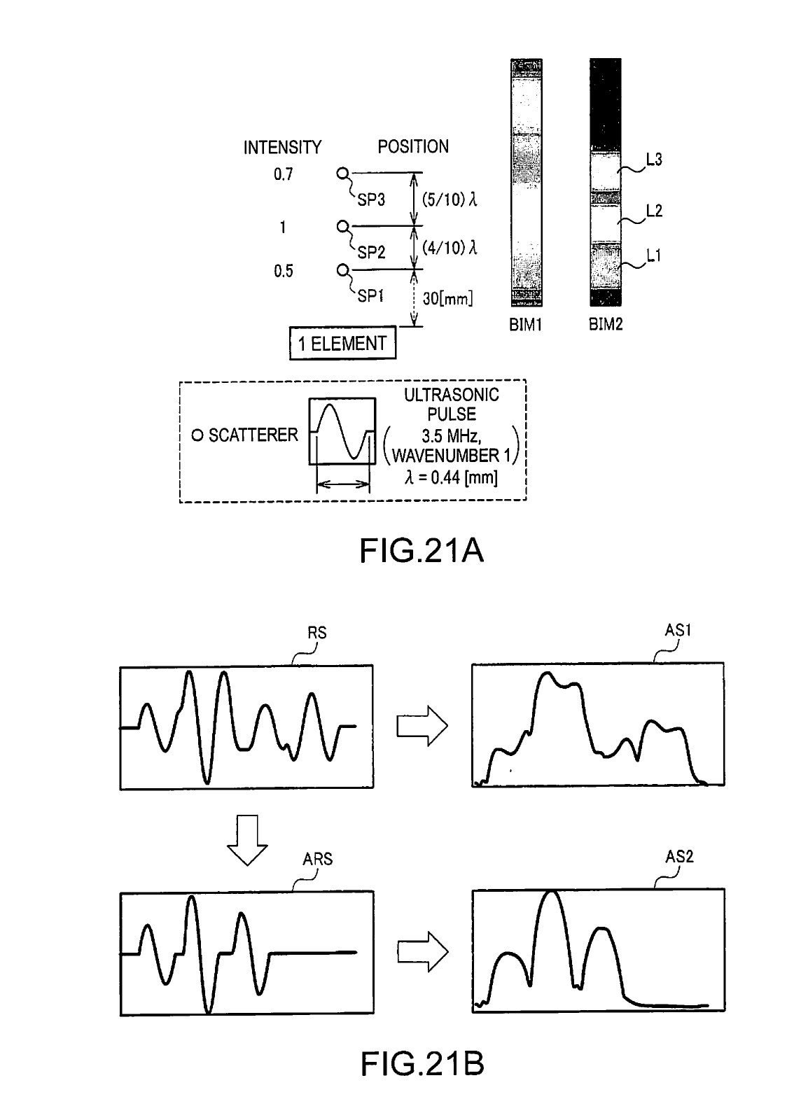

FIGS. 21A and 21B show an example of a measurement result according to the present embodiment. In this example, as shown in FIG. 21A, an ultrasonic wave pulse having a wavelength A of 0.44 mm, the wavenumber of 1, and the frequency of 3.5 MHz is transmitted to a subject.

In the example of FIG. 21A, it is assumed that the upward direction of the drawing is the depth direction of the subject, and there are three point scatterers (SP1 to SP3) in the subject. Also, it is assumed that the ultrasonic transducer element and the point scatterer SP1 are separated from each other by 30 mm, the point scatterer SP1 and the point scatterer SP2 are separated from each other by (4/10)λ, and the point scatterer SP2 and the point scatterer SP3 are separated from each other by (5/10)λ. Furthermore, the reflection intensity from the point scatterer SP1 is 0.5, the reflection intensity from the point scatterer SP2 is 1.0, and the reflection intensity from the point scatterer SP3 is 0.7.

In this case, when a B-mode image is generated only using the phase inversion method and the filter method, an image BIM1 of the same figure is generated. In this image BIM1, reflective waves from the point scatterers overlap each other, the tone of the entire image hardly changes, and thus it is difficult to specify the detailed positions of the three point scatterers.

On the other hand, when a B-mode image is generated based on the above-described reconstructed wave of the present embodiment, an image BIM2 of the same figure is generated. It is clear from this image BIM2 that a color layer L1 corresponds to the reflection from the point scatterer SP1, a color layer L2 corresponds to the reflection from the point scatterer SP2, and a color layer L3 corresponds to the reflection from the point scatterer SP3. That is, the distance resolution is improved as compared with the case in which only the phase inversion method and the filter method are used.

Furthermore, the processing unit 130 may perform envelope detection processing on the reconstructed signal obtained after the conversion processing.

As shown in, for example, FIG. 21B, a waveform AS1 is obtained by performing envelope detection processing on the waveform of the received signal RS subjected to the processing using a phase inversion method and a filter method. In the waveform AS1, two large peaks can be recognized, but it is difficult to determine that the subject includes three point scatterers.

In contrast, a waveform AS2 is obtained by performing the processing of the present embodiment on the received signal RS to obtain a waveform ARS and performing envelope detection processing on the waveform ARS. In the waveform AS2, three peaks are recognized, and it is easily possible to determine that they are the peaks corresponding to the reflections from the point scatterers. Furthermore, as compared with the waveform AS1, the positions of the peaks of the waveform AS2 are closer to the distribution of the point scatterers in the actual subject.

Accordingly, it is possible, for example, to display, on the display unit 300, waveform data from which a user can easily understand the measurement result.

### 4. Ultrasonic Transducer Element

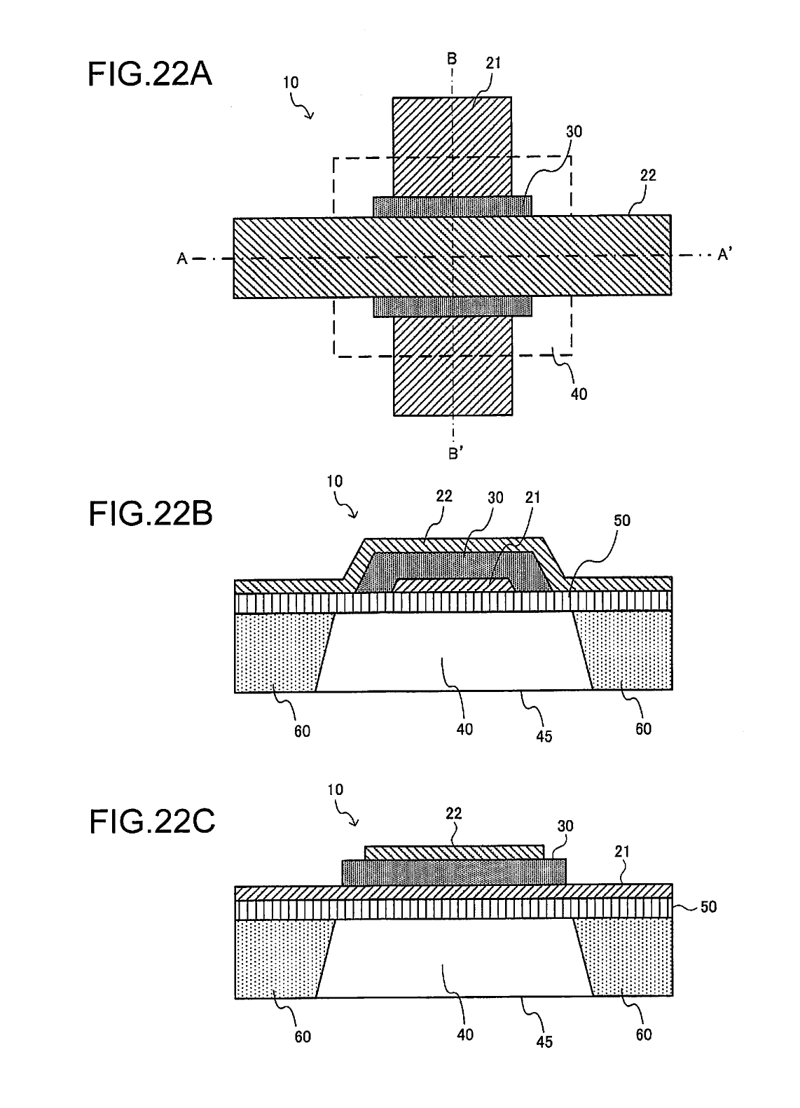

FIGS. 22A to 22C show an example of a configuration of an ultrasonic transducer element 10 of the ultrasonic transducer device. This ultrasonic transducer element 10 includes a vibrating film (membrane, supporting member) 50 and a piezoelectric element section. The piezoelectric element section includes a first electrode layer (lower electrode) 21, a piezoelectric material layer (piezoelectric material film) 30, and a second electrode layer (upper electrode) 22.

FIG. 22A is a plan view of the ultrasonic transducer element 10 that is formed on a substrate (silicon substrate) 60, viewed in the direction perpendicular to the substrate 60 on the element forming surface side. FIG. 22B is a cross-sectional view taken along the line A-A′ of FIG. 22A. FIG. 22C is a cross-sectional view taken along the line B-B′ of FIG. 22A.

The first electrode layer 21 is made from, for example, a metal thin film, and is formed on the upper layer of the vibrating film 50. This first electrode layer 21 may extend to the outside of an element forming region as shown in FIG. 22A, and may be an interconnect connected to an adjacent ultrasonic transducer element 10.

The piezoelectric material layer 30 is made from, for example, a PZT (zirconate titanate) thin film, and is provided so as to cover at least a part of the first electrode layer 21. Note that the material of the piezoelectric material layer 30 is not limited to PZT and may be made from, for example, lead titanate (PbTiO₃), lead zirconate (PbZrO₃), lead lanthanum titanate ((Pb, La)TiO₃), or the like.

The second electrode layer 22 is made from, for example, a metal thin film, and is provided so as to cover at least a part of the piezoelectric material layer 30. This second electrode layer 22 extends to the outside of the element forming region as shown in FIG. 22A, and may be an interconnect connected to an adjacent ultrasonic transducer element 10.

The vibrating film (membrane) 50 has a two-layer structure of, for example, a SiO₂ thin film and a ZrO₂ thin film, and is provided so as to cover the opening 40. This vibrating film 50 supports the piezoelectric material layer 30 and the first and second electrode layers 21 and 22, and vibrates in accordance with the expansion and contraction of the piezoelectric material layer 30, so as to be able to generate an ultrasonic wave.

The opening 40 is formed by performing etching such as reactive ion etching (RIE) on the rear surface (on which no element is formed) of the substrate 60 (silicon substrate). The resonance frequency of the ultrasonic wave is determined depending on the size of an open section 45 of the opening 40, and the ultrasonic wave is emitted to the piezoelectric material layer 30 side (in the direction from back to front of FIG. 22A).

The lower electrode (first electrode) of the ultrasonic transducer element 10 is formed by the first electrode layer 21, and the upper electrode (second electrode) thereof is formed by the second electrode layer 22. Specifically, the section of the first electrode layer 21 that is covered with the piezoelectric material layer 30 forms the lower electrode, and the section of the second electrode layer 22 that covers the piezoelectric material layer 30 forms the upper electrode. That is, the piezoelectric material layer 30 is provided between the lower electrode and the upper electrode.

### 5. Ultrasonic Transducer Device

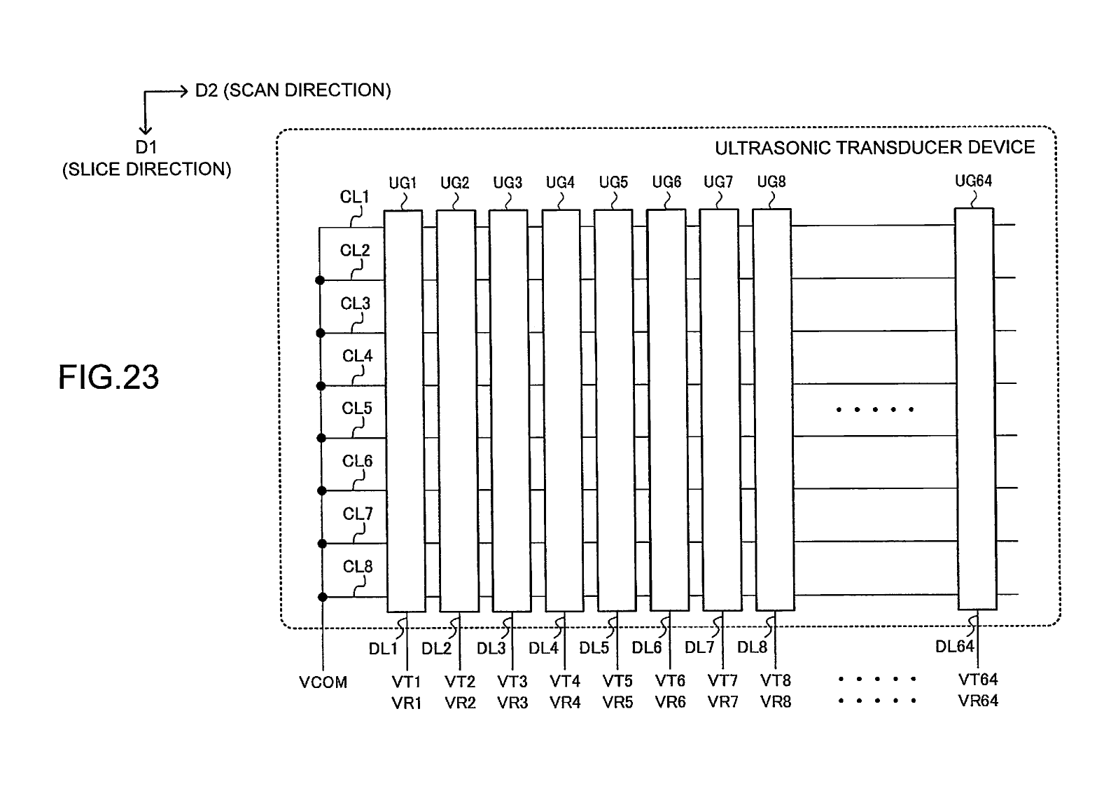

FIG. 23 shows an example of a configuration of an ultrasonic transducer device (component chip). The ultrasonic transducer device according to the present configuration example includes a plurality of ultrasonic transducer element groups UG1 to UG64, drive electrode lines DL1 to DL64 (in a broad sense, first to n-th drive electrode lines, where n is an integer of 2 or greater), and common electrode lines CL1 to CL8 (in a broad sense, first to m-th common electrode line, where m is an integer of 2 or greater). Note that the number (n) of the drive electrode lines or the number (m) of the common electrode line are not limited to the numbers shown in FIG. 23.

The plurality of ultrasonic transducer element groups UG1 to UG64 are arranged in sixty-four lines in a second direction D2 (scan direction). Each of the ultrasonic transducer element groups UG1 to UG64 has a plurality of ultrasonic transducer elements that are arranged in a first direction D1 (slice direction).

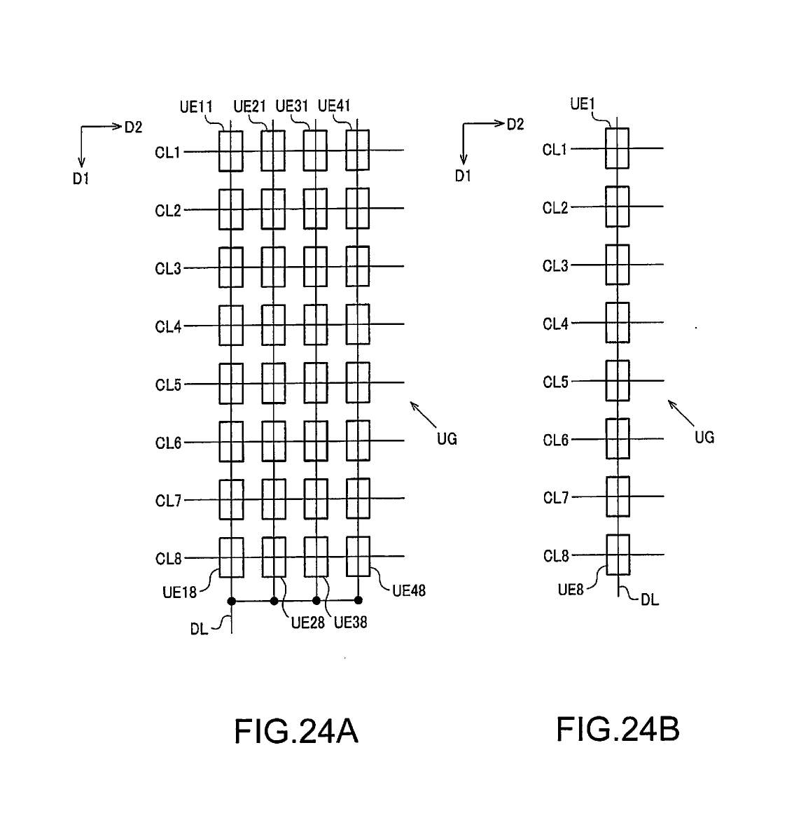

FIG. 24A shows an example of an ultrasonic transducer element group UG (one of UG1 to UG64). In FIG. 24A, an ultrasonic transducer element group UG is constituted by the first to fourth element lines. The first element line is constituted by ultrasonic transducer elements UE11 to UE18 that are arranged in the first direction D1, and the second element line is constituted by ultrasonic transducer elements UE21 to UE28 that are arranged in the first direction D1. The same applies to the third element line (UE31 to UE38) and the fourth element line (UE41 to UE48). A drive electrode line DL (one of DL1 to DL64) is connected in common to the first to fourth element lines. Furthermore, the common electrode lines CL1 to CL8 are connected to the ultrasonic transducer elements of the first to fourth element lines.

Also, the ultrasonic transducer element group UG of FIG. 24A constitute one channel of the ultrasonic transducer device. That is, the drive electrode line DL corresponds to a drive electrode line of one channel, and a transmission signal for one channel from the transmission circuit is input to the drive electrode line DL. Furthermore, a received signal for one channel from the drive electrode line DL is output from the drive electrode line DL. Note that the number of element lines constituting one channel is not limited to four as shown in FIG. 24A, and may be less or more than four. For example, as shown in FIG. 24B, one element line may constitute one channel.

As shown in FIG. 23, the drive electrode lines DL1 to DL64 (first to n-th drive electrode lines) are arranged in the first direction D1. The j-th drive electrode line DLj (j-th channel) (where j is an integer of 1≦j≦n) among the drive electrode lines DL1 to DL64 is connected to the first electrode (for example, the lower electrode) of an ultrasonic transducer element of the j-th ultrasonic transducer element group UGj.

During a transmission time period in which an ultrasonic wave is emitted, transmission signals VT1 to VT64 are supplied to the ultrasonic transducer elements via the drive electrode lines DL1 to DL64. Furthermore, during a reception time period in which ultrasonic echo signals are received, received signals VR1 to VR64 from the ultrasonic transducer elements are output via the drive electrode lines DL1 to DL64.

The common electrode lines CL1 to CL8 (first to m-th common electrode lines) are arranged in the second direction D2. The second electrodes of the ultrasonic transducer elements are each connected to the corresponding one of the common electrode lines CL1 to CL8. Specifically, as shown in FIG. 23 for example, the i-th common electrode line CLi (where i is an integer of 1≦i≦m) among the common electrode lines CL1 to CL8 is connected to the second electrodes (for example, the upper electrodes) of the ultrasonic transducer elements arranged in the i-th row.

A common voltage VCOM is supplied to the common electrode lines CL1 to CL8. This common voltage VCOM only needs to be a constant direct-current voltage, and is not necessarily 0V, namely, a ground electric potential (ground potential).

During the transmission time period, a voltage corresponding to a difference between a transmission signal voltage and a common voltage is applied to the ultrasonic transducer elements, and an ultrasonic wave with a predetermined frequency is emitted.

Note that the arrangement of the ultrasonic transducer elements is not limited to the matrix arrangement shown in FIG. 23, and may be a so-called staggered arrangement or the like.

Furthermore, FIGS. 24A and 24B illustrate a case where one ultrasonic transducer element is used as both a transmission element and a reception element, but the present embodiment is not limited to the case. For example, ultrasonic transducer elements for transmission elements and ultrasonic transducer elements for reception elements are provided in a separate manner, and may be arranged in an array.

Note that the ultrasonic measurement device, the ultrasonic imaging device, or the like of the present embodiment may be realized by a program that performs a part or most part of processing. In this case, by a processor such as a CPU executing the program, the ultrasonic measurement device, the ultrasonic imaging device, or the like of the present embodiment is realized. Specifically, a program stored in a non-transitory information storage device is read, and the read program is executed by a processor such as a CPU. In this context, the information storage device (a computer readable device) is a device in which a program, data, and the like are stored, and whose functions can be realized by an optical disc (such as a DVD or CD), an HDD (hard disk drive), a memory (such as a card-type memory or a ROM), or the like. Also, a processor such as a CPU executes various types of processing of the present embodiment based on the programs (data) stored in the information storage device. That is, the information storage device has stored therein a program for causing a computer (device including an operation unit, a processing unit, a storage unit, and an output unit) to function as the components of the embodiments (programs for causing a computer to execute processing of the components).

Furthermore, the ultrasonic measurement device, the ultrasonic imaging device, and the like of the embodiments may include a processor and a memory. Here, the processor may be, for example, a CPU (Central Processing Unit). However, the processor is not limited to the CPU, and may employ various types of processors such as a GPU (Graphics Processing Unit) and a DSP (Digital Signal Processor). Furthermore, the processor may be a hardware circuit using an ASIC (Application Specific Integrated Circuit). Furthermore, the memory stores computer readable commands, and by the commands being executed by the processor, the components of the ultrasonic measurement device, the ultrasonic imaging device, or the like of the present embodiment will be realized. The memory in this context may be a semiconductor memory, such as an SRAM (Static Random Access Memory) or a DRAM (Dynamic Random Access Memory), a register, a hard disk, or the like. Furthermore, the commands in this context may be a command set of commands constituting the program, or commands to instruct the hardware circuit of the processor to operate.

As described above, the embodiments have been described in detail, but it can readily be appreciated to those skilled in the art that various modifications are possible without substantially departing from the novel features and effects of the invention. Therefore, all the modifications are included in the scope of the invention. For example, a term that is used in the specification and the drawings at least once together with a different term having a broader or the same meaning can be replaced with this different term in any place of the specification or the drawings. Furthermore, the configurations and operations of the ultrasonic measurement device, the ultrasonic imaging device, and the ultrasonic measurement method are not limited to those described in the present embodiment, and various modifications are possible.

The entire disclosure of Japanese Patent Application No. 2014-222251 filed on Oct. 31, 2014 is expressly incorporated by reference herein.

What is claimed is:

1. An ultrasonic measurement device comprising:
a transmission processing unit that performs processing for transmitting an ultrasonic wave to a subject;
a reception processing unit that performs processing for receiving an ultrasonic echo of the transmitted ultrasonic wave; and
a processing unit that performs processing on a received signal from the reception processing unit,
wherein the processing unit performs, on the received signal, processing for specifying reflection intensities from point scatterers in the subject based on M basis waves (where M is an integer of 2 or more) at least two of which have phases shifted from each other with a phase difference smaller than a phase difference that corresponds to a pulse width of a transmission pulse signal transmitted by the transmission processing unit or a pulse width of a received signal in response to the transmission pulse signal.

2. The ultrasonic measurement device according to claim 1, further comprising:
a storage unit in which respective pieces of basis wave data on the M basis waves are stored in association with M measurement points that are set in a depth direction of the subject.

3. The ultrasonic measurement device according to claim 1,
wherein a phase difference between an i-th basis wave (where i is an integer of 1≦i≦M) and an (i+1)-th basis wave of the M basis waves is smaller than a phase distance that corresponds to the pulse width of the transmission pulse signal or the pulse width of the received signal.

4. The ultrasonic measurement device according to claim 1,
wherein the i-th basis wave (where i is an integer of 1≦i≦M) of the M basis waves is a waveform that corresponds to a received signal of the ultrasonic wave from an i-th point scatterer arranged at an i-th measurement point, and
the (i+1)-th basis wave of the M basis waves is a waveform that corresponds to a received signal of the ultrasonic wave from an (i+1)-th point scatterer arranged at an (i+1)-th measurement point, which is farther away from a transmission point of the ultrasonic wave than the i-th measurement point.

5. The ultrasonic measurement device according to claim 1,
wherein the processing unit obtains the reflection intensities by performing deconvolution processing of the M basis waves on the received signal.

6. The ultrasonic measurement device according to claim 1,
wherein a set distance between measurement points in a first depth range of the subject is smaller than a set distance between measurement points in a second depth range of the subject.

7. The ultrasonic measurement device according to claim 1,
wherein the processing unit performs the processing for specifying reflection intensities in the first depth range of the subject, and does not perform the processing for specifying reflection intensities in the second depth range of the subject.

8. The ultrasonic measurement device according to claim 1,
wherein the processing unit performs processing for generating a B-mode image based on the reflection intensities specified by the specifying processing.

9. The ultrasonic measurement device according to claim 1,
wherein each of the M basis waves is a waveform obtained by shifting a phase of the transmission pulse signal.

10. An ultrasonic imaging device comprising:
the ultrasonic measurement device according to claim 1; and
a display unit that displays image data for display that is created based on the reflection intensities.

11. An ultrasonic imaging device comprising:
the ultrasonic measurement device according to claim 2; and
a display unit that displays image data for display that is created based on the reflection intensities.

12. An ultrasonic imaging device comprising:
the ultrasonic measurement device according to claim 3; and
a display unit that displays image data for display that is created based on the reflection intensities.

13. An ultrasonic imaging device comprising:
the ultrasonic measurement device according to claim 4; and
a display unit that displays image data for display that is created based on the reflection intensities.

14. An ultrasonic imaging device comprising:
the ultrasonic measurement device according to claim 5; and
a display unit that displays image data for display that is created based on the reflection intensities.

15. An ultrasonic imaging device comprising:
the ultrasonic measurement device according to claim 6; and
a display unit that displays image data for display that is created based on the reflection intensities.

16. An ultrasonic imaging device comprising:
the ultrasonic measurement device according to claim 7; and
a display unit that displays image data for display that is created based on the reflection intensities.

17. An ultrasonic imaging device comprising:
the ultrasonic measurement device according to claim 8; and
a display unit that displays image data for display that is created based on the reflection intensities.

18. An ultrasonic imaging device comprising:
the ultrasonic measurement device according to claim 9; and
a display unit that displays image data for display that is created based on the reflection intensities.

19. An ultrasonic measurement method comprising:
performing processing for transmitting an ultrasonic wave to a subject;
performing processing for receiving an ultrasonic echo reflected by the transmitted ultrasonic wave so as to obtain a received signal; and
performing, on the received signal, processing for specifying reflection intensities from point scatterers in the subject based on M basis waves (where M is an integer of 2 or more) that have phases shifted from each other with a phase difference smaller than a phase difference that corresponds to a pulse width of a transmission pulse signal transmitted by the transmission processing unit or a pulse width of a received signal in response to the transmission pulse signal.
